(19) United States
(12) Patent Application Publication
(10) Pub. No.: US 2025/0005712 A1
(43) Pub. Date: Jan. 2, 2025

(54) SYSTEMS AND METHODS FOR GENERATING A CORRECTED PLANAR SCINTIGRAPHY IMAGE (CPSI)

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ross Schmidtlein, New York, NY (US); Yuseheng Xu, Virginia Beach, VA (US); Andrzej Krol, Fayetteville, NY (US); Howard Gifford, Houston, TX (US)

(21) Appl. No.: 18/707,555

(22) PCT Filed: Nov. 4, 2022
(86) PCT No.: PCT/US2022/049034

§ 371 Date: May 3, 2024

Related U.S. Application Data
(60) Provisional application No. 63/276,504, filed on Nov. 5, 2021.

(51) Int. Cl. G06T 5/10; A61B 6/00; A61B 6/40; A61B 6/42; G06T 5/70; G06T 11/00

(52) U.S. Cl. CPC G06T 5/10; A61B 6/4057; A61B 6/4258; A61B 6/5258; G06T 5/70; G06T 11/006; G06T 2207/10081; G06T 2207/10108; G06T 2207/20052; G06T 2207/30004; G06T 2210/41; G06T 2211/40

(57) ABSTRACT

Described embodiments provide systems and methods for generating a corrected planar scintigraphy image (CPSI) corrected for image artifacts. A computing system can obtain a plurality of planar scintigraphy images of a subject. The plurality of planar scintigraphy images may contain image artifacts caused by one or more physical processes. The computing system may generate a CPSI corrected for the image artifacts by applying a planar scintigraphy image reconstruction model to the plurality of planar scintigraphy images. The planar scintigraphy image reconstruction model may comprise a non-negativity constraint and be based on a regularization term and a fidelity term. The computing system may present the CPSI for evaluation of a condition of the subject. Presenting the CPSI may comprise at least one of transmitting the CPSI to a computing device or displaying the CPSI on a display screen.

### REFERENCE TO RELATED APPLICATIONS

This application claims priority to U.S. Provisional Patent Application No. 63/276,504 filed Nov. 5, 2021, the entirety of which is incorporated herein by reference.

### FIELD OF THE DISCLOSURE

The present disclosure is directed to reconstructing and/or generating a corrected planar scintigraphy image (CPSI) corrected for (e.g., without or with reduced), image artifacts based on a plurality of planar scintigraphy images.

### BACKGROUND OF THE DISCLOSURE

The following description of the background of the present technology is provided simply as an aid in understanding the present technology and is not admitted to describe or constitute prior art to the present technology.

Certain nuclear medical imaging methods, such as scintigraphy, can be used for imaging a bio-distribution of a molecular target by using radiotracers and gamma ray detectors. A pair of opposing gamma ray detectors (e.g., equipped with parallel-hole lead or tungsten collimators) can be used to obtain one or more medical images of a subject. The obtained medical images may contain image noise and image artifacts. Certain techniques, such as post-filtering the medical images, can reduce the image noise at the expense of spatial resolution. However, said techniques are insufficient in removing the aforementioned image artifacts from the medical images.

### SUMMARY

This Summary is provided to introduce a selection of concepts in a simplified form that are further described below in the Detailed Description. This Summary is not intended to identify key features or essential features, nor is it intended to limit the scope of the claims included herewith.

The present disclosure is directed towards systems and methods for generating and/or reconstructing a CPSI (or other medical images) with certain image artifacts reduced or eliminated (e.g., artifacts caused by gamma ray attenuation, gamma ray collimator penetration, gamma ray scatter, and/or other processes) by using (or according to) a plurality of planar scintigraphy images. The systems and methods, for instance, can be based on a planar scintigraphy image reconstruction model/approach for generating a CPSI according to an anterior planar scintigraphy image or a posterior planar scintigraphy image (e.g., an anterior/posterior (A/P) image pair (conjugate views)). The planar scintigraphy image reconstruction model can be based on a physical model of a two-view single photon emission computed tomography (SPECT) system, in which the physical properties of a plurality of gamma ray detectors (or other types of detectors) are considered (e.g., to reconstruct a planar projection, such as the CPSI).

In one aspect, the present disclosure is directed to a computing system for generating and/or reconstructing a CPSI corrected for image artifacts (e.g., caused by gamma ray attenuation, gamma ray collimator penetration, gamma ray scatter, and/or other processes) based on (or according to) a plurality of planar scintigraphy images. The system may comprise one or more processors and a non-transitory computer-readable medium having instructions stored thereon. The one or more processors can execute the instructions stored on the non-transitory computer-readable medium. Upon execution of the instructions by the one or more processors, the instructions may cause the system to obtain a plurality of planar scintigraphy images of a subject. The plurality of planar scintigraphy images may contain/include image artifacts caused by one or more physical processes. The instructions may cause the system to generate, by the one or more processors, a CPSI corrected for (e.g., without) the image artifacts by applying a planar scintigraphy image reconstruction model to the plurality of planar scintigraphy images. The planar scintigraphy image reconstruction model may comprise a non-negativity constraint. The planar scintigraphy image reconstruction model can be based on a regularization term and a fidelity term. The instructions may cause the system to present, by the one or more processors, the CPSI for evaluation of a condition of the subject. Presenting the CPSI may comprise at least one of transmitting the CPSI to a computing device or displaying the CPSI on a display screen.

In some embodiments, the plurality of planar scintigraphy images may comprise an anterior planar scintigraphy image or a posterior planar scintigraphy image. In certain embodiments, the one or more physical processes may comprise gamma ray attenuation, gamma ray collimator penetration, or gamma ray scatter. In some embodiments, obtaining the plurality of planar scintigraphy images may comprise using a plurality of gamma ray detectors to generate the plurality of planar scintigraphy images. In certain embodiments, the regularization term may correspond to a total variation regularization for controlling noise. In some embodiments, the regularization term may correspond to (or be associated with) other types of regularization approaches for controlling noise (e.g., tight framelets based on a redundant multiscale discrete cosine transform, deep learning approaches, and/or other types of approaches for controlling noise). In certain examples, the regularization term may include or correspond to other types of regularization for controlling noise, such as deep learning, sparse representation, quadratic, and/or other types. In some embodiments, the planar scintigraphy image reconstruction model may comprise a minimization operation based on the regularization term, the fidelity term, and the non-negativity constraint. The minimization operation can be based on a divergence norm and a regularization parameter (λ).

In some embodiments, the planar scintigraphy image reconstruction model may be based on a two-view single photon emission computed tomography (SPECT) physical model. The two-view SPECT physical model may comprise an anterior view and a posterior view. In certain embodiments, generating the CPSI may comprise estimating an anterior/posterior (A/P) projection of activity bio-distribution using the two-view SPECT physical model as a constraint. The two-view SPECT physical model may be determined according to ∫_{J}_{³}K(x; y)ƒ(y)dy=g (x), x∈J³, wherein x:=(x₁, x₂, x₃), y:=(y₁, y₂, y₃), the g(x) describes the plurality of planar scintigraphy images, the ƒ(y) represents/describes a 3D biodistribution, and the K(x; y) describes a kernel of a region J:=[α, b]. In some embodiments, the K(x; y) can be related to an attenuation map derived from computed tomography (CT). In one example, the K (x; y) may be determined according to K (x; y):=δ(x₁−y₁, x₂−y₂)e^{-∫}^{_{y3}}^{^{x3}}^{μ(y1,y2,t)dt} and/or other types of kernels that include accurate physical models. The μ(y), y∈J³ can describe an attenuation map derived from CT. In one example, the K(x; y) can be determined according to

$$K ( x ; y ) = \frac{1}{2 {πσ}_{x}^{2} ( {y}_{3} )} \times {e}^{- ( {( \frac{{x}_{1} - {y}_{1}}{\sqrt{2} {σ}_{x} ( {y}_{3} )} )}^{2} + {( \frac{{x}_{2} - {y}_{2}}{\sqrt{2} {σ}_{x} ( {y}_{3} )} )}^{2} + {\int }_{{x}_{3}}^{{y}_{3}} μ ( {y}_{1} , {y}_{2} , t ) dt )} ,$$

wherein a resolution loss is dependent of y₃ and invariant in the y_{1,2} plane, and a Gaussian blur with a variance of σₓ²(y₃) is used. In certain embodiments, applying the planar scintigraphy image reconstruction model may comprise determining ƒ̅(y₁, y₂)∫_{J}, ƒ(y₁, y₂, y₃)dy₃ subject to:

$${min}_{f} \{ {‖ {\int }_{{J}^{3}} K ( \cdot ; y ) f ( y ) dy - g ( \cdot ) ‖}_{KL} + λ {‖ f ‖}_{TV ( {J}^{3} )} + {ι}_{+} ( f ) \} ,$$

wherein the ƒ̅(y₁, y₂) describes the CPSI, and the ∥∫_{J}_{³}K(·; y)ƒ(y)dy−g(·)∥_{KL} denotes or corresponds to a divergence norm corresponding to the fidelity term. In certain embodiments, the fidelity term may include or correspond to other types of norms or semi-norms, such as an L1-norm, a KL-norm, and/or other types of norms or semi-norms. In some embodiments, the λ|ƒ∥_{TV(J}_{³}₎ denotes corresponds to a total variation regularization for controlling noise corresponding to the regularization term. In some embodiments, the regularization term may include or correspond to other types of regularization approaches, such as quadratic, sparse representation, and deep learning. In certain embodiments, the l₊(ƒ) may correspond to an indicator function imposing the non-negativity constraint.

Applying the planar scintigraphy image reconstruction model may comprise determining ƒ̅(y₁, y₂)=∫_{J} ƒ(y₁, y₂, y₃)dy₃ subject to:

$${min}_{f} \{ {‖ {\int }_{J} {e}^{- {\int }_{{y}_{3}}^{{x}_{3}} μ ( {y}_{1} , {y}_{2} , t ) dt} f ( {y}_{1} , {y}_{2} , {y}_{3} ) {dy}_{3} - g ( {y}_{1} , {y}_{2} ) ‖}_{KL} +$$

$$λ {‖ f ‖}_{TV ( {J}^{3} )} + {ι}_{+} ( f ) \} ,$$

wherein the ƒ̅(y₁, y₂) describes the CPSI, and the ∥∫_{J}e^{-∫}^{_{y3}}^{^{x3}}^{μ(y1, y2, t)dt}ƒ(y₁, y₂, y₃)dy₃−g(y₁, y₂)∥_{KL} denotes or corresponds to a divergence norm corresponding to the fidelity term. In certain embodiments, the fidelity term may include or correspond to other types of norms, such as an L1-norm, a KL-norm, and/or other types of norms or semi-norms. In some embodiments, the λ∥ƒ∥_{TV(J}_{³}₎ denotes or corresponds to a total variation regularization for controlling noise corresponding to the regularization term. In some embodiments, the regularization term may include or correspond to other types of regularization approaches, such as quadratic, sparse representation, and deep learning. In certain embodiments, the l₊(ƒ) may correspond to an indicator function imposing the non-negativity constraint. In some embodiments, the planar scintigraphy image reconstruction model can be discretized according to f̅=Σ_{y}_{₃}ƒ(y), subject to:

$${min}_{f} \{ {‖ Kf - g ‖}_{KL} + λ {‖ {B}_{3} f ‖}_{1} + {ι}_{+} ( f ) \} ,$$

∥Kƒ−g∥_{KL}=Kƒ−g log (Kƒ), wherein the B₃ denotes or corresponds to a three dimensional first order gradient block matrix. In some embodiments, the instructions may further cause the computing system to apply or solve the discretized planar scintigraphy image reconstruction model using a fixed point algorithm with higher order total variation regularization (HOTV) according to:

$$\{ \begin{matrix}{f}^{k + 1} = {prox}_{{ι}_{+}} \{ {f}^{k} - S [ {K}^{T} ( 1 - \frac{g}{{Kf}^{k}} ) + {B}_{3}^{T} {b}^{k} + {C}_{3}^{T} {c}^{k} ] \} \\ {b}^{k + 1} = {ρ}_{1} ( I - {prox}_{\frac{{λ}_{1}}{{ρ}_{1}} {φ}_{1}} ) ( \frac{{b}^{k}}{ρ} + {B}_{3} ( 2 {f}^{k + 1} - {f}^{k} ) ) \\ {c}^{k + 1} = {ρ}_{2} ( I - {prox}_{\frac{{λ}_{2}}{{ρ}_{2}} {φ}_{2}} ) ( \frac{{c}^{k}}{ρ} + {C}_{3} ( 2 {f}^{k + 1} - {f}^{k} ) )\end{matrix} ,$$

wherein the b denotes (or corresponds to) a first subgradient term, the c denotes (or corresponds to) a second subgradient term, the C₃ denotes (or corresponds to) a three dimensional second order gradient block matrix, the φ₁ denotes (or corresponds to) a first order isotropic total variation norm, the φ₂ denotes (or corresponds to) a second order isotropic total variation norm, the λ₁ denotes (or corresponds to) a first regularization parameter, the λ₂ denotes (or corresponds to) a second regularization parameter, the S denotes (or corresponds to) a preconditioner, the ρ₁ denotes (or corresponds to) a first algorithmic parameter, and the ρ₂ denotes (or corresponds to) a second algorithmic parameter. In some embodiments, the instructions may further cause the computing system to apply or solve the discretized planar scintigraphy image reconstruction model using other approaches, such as alternating direction method of multipliers (ADMM), Chambolle-Pock, and/or other approaches.

In one aspect, the present disclosure is directed to a method for generating and/or reconstructing a CPSI corrected for (e.g., without) image artifacts (e.g., caused by gamma ray attenuation, gamma ray collimator penetration, gamma ray scatter, and/or other processes) based on (or according to) a plurality of planar scintigraphy images. A computing system may obtain a plurality of planar scintigraphy images of a subject. The plurality of planar scintigraphy images may contain image artifacts caused by one or more physical processes. The computing system may generate a CPSI corrected for (e.g., without) the image artifacts by applying a planar scintigraphy image reconstruction model to the plurality of planar scintigraphy images. The planar scintigraphy image reconstruction model may comprise a non-negativity constraint. The planar scintigraphy image reconstruction model may be based on a regularization term and a fidelity term. The computing system may present the CPSI for evaluation of a condition of the subject. Presenting the CPSI may comprise at least one of transmitting the CPSI to a computing device or displaying the CPSI on a display screen.

In some embodiments, the plurality of planar scintigraphy images may comprise an anterior planar scintigraphy image or a posterior planar scintigraphy image. In certain embodiments, the one or more physical processes may comprise gamma ray attenuation, gamma ray collimator penetration, or gamma ray scatter. In some embodiments, obtaining the plurality of planar scintigraphy images may comprise using a plurality of gamma ray detectors to generate the plurality of planar scintigraphy images. In certain embodiments, the regularization term may correspond to a total variation regularization for controlling noise. In some embodiments, the regularization term may correspond to (or be associated with) other types of regularization approaches for controlling noise (e.g., tight framelets based on a redundant multiscale discrete cosine transform, deep learning approaches, and/or other types of approaches for controlling noise). In certain examples, the regularization term may include or correspond to other types of regularization for controlling noise, such as deep learning, sparse representation, quadratic, and/or other types. In some embodiments, the planar scintigraphy image reconstruction model may comprise a minimization operation based on the regularization term, the fidelity term, and the non-negativity constraint. The minimization operation can be based on a divergence norm and a regularization parameter (λ). In certain embodiments, the method may include determining, according to the generated CPSI, a dosage of radiation administered to the subject that minimizes a risk of toxicity to non-cancerous tissue, while optimizing treatment for cancerous tissue.

In some embodiments, the planar scintigraphy image reconstruction model may be based on a two-view single photon emission computed tomography (SPECT) physical model. The two-view SPECT physical model may comprise an anterior view and a posterior view. In certain embodiments, generating the CPSI may comprise estimating an anterior/posterior (A/P) projection of activity bio-distribution using the two-view SPECT physical model as a constraint. In some embodiments, the method may include determining the two-view SPECT physical model according to ∫_{J}³ K(x; y)ƒ(y)dy=g(x), x∈J³, wherein x:=(x₁, x₂, x₃), y:=(y₁, y₂, y₃), the g(x) describes the plurality of planar scintigraphy images, the ƒ(y) represents a 3D biodistribution and the K(x; y) describes a kernel of a region J:=[α, b]. In some embodiments, the K(x; y) can be determined according to an attenuation map derived from CT. In some embodiments, the method may include determining the K(x; y) according to K(x; y)=δ(x₁−y₁, x₂−y₂)e⁻^{_{∫y3}}^{^{x3}}^{μ(y1,y2,t)dt} and/or other kernels that include accurate physical models (e.g., physical models that include resolution loss, collimator penetration, and/or scatter estimation). The μ(y), y∈J³ may describe an attenuation map derived from CT. In certain embodiments, applying the planar scintigraphy image reconstruction model may comprise determining ƒ̅(y₁, y₂)=∫_{J}, f (y₁, y₂, y₃)dy₃ subject to:

$${min}_{f} \{ {‖ {\int }_{{J}^{3}} K ( \cdot ; y ) f ( y ) dy - g ( \cdot ) ‖}_{KL} + λ {‖ f ‖}_{TV ( {J}^{3} )} + {ι}_{+} ( f ) \} ,$$

wherein the ƒ̅(y₁, y₂) describes the CPSI, the ∥∫_{J3}K(·;y)ƒ(y)dy−g(·)∥_{KL} denotes or corresponds to a divergence norm corresponding to the fidelity term, the λ∥ƒ∥_{TV(J}_{³}₎ denotes corresponds to a total variation regularization for controlling noise corresponding to the regularization term, and the l₊(f) corresponds to an indicator function imposing the non-negativity constraint.

Applying the planar scintigraphy image reconstruction model may comprise determining ƒ̅(y₁, y₂)=∫_{J} ƒ(y₁, y₂, y₃)dy₃ subject to:

$${min}_{f} \{ {‖ {\int }_{J} {e}^{- {\int }_{{y}_{3}}^{{x}_{3}} μ ( {y}_{1} , {y}_{2} , t ) dt} f ( {y}_{1} , {y}_{2} , {y}_{3} ) {dy}_{3} - g ( {y}_{1} , {y}_{2} ) ‖}_{KL} +$$

$$λ {‖ f ‖}_{TV ( {J}^{3} )} + {ι}_{+} ( f ) \} ,$$

wherein the ƒ̅(y₁, y₂) describes the CPSI, and the ∥∫_{J}e^{-∫}^{_{y3}}^{^{x3}}^{μ(y1,y2,t)dt}ƒ(y₁, y₂, y₃)dy₃−g(y₁, y₂)∥_{KL} denotes corresponds to a divergence norm corresponding to the fidelity term. In certain embodiments, the fidelity term may include or correspond to other types of norms, such as an L1-norm, a KL-norm, and/or other types of norms. In some embodiments, the λ∥ƒ∥_{TV(J}_{³}₎ denotes corresponds to a total variation regularization for controlling noise corresponding to the regularization term. In some embodiments, the regularization term may include or correspond to other types of regularization approaches. In certain embodiments, the l₊(ƒ) may correspond to an indicator function imposing the non-negativity constraint. In some embodiments, the method may include discretizing the planar scintigraphy image reconstruction model according to ƒ̅=Σ_{y}_{₃}ƒ(y), subject to:

$${min}_{f} \{ {‖ Kf - g ‖}_{KL} + λ {‖ {B}_{3} f ‖}_{1} + {ι}_{+} ( f ) \} , {‖ Kf - g ‖}_{KL} = Kf - g log ( Kf ) ,$$

wherein the B₃ denotes (or corresponds to) a three dimensional first order gradient block matrix. In some embodiments, the method may include applying or solving the discretized planar scintigraphy image reconstruction model using a fixed point algorithm with higher order total variation regularization (HOTV) according to:

$$\{ \begin{matrix}{f}^{k + 1} = {prox}_{{ι}_{+}} \{ {f}^{k} - S [ {K}^{T} ( 1 - \frac{g}{{Kf}^{k}} ) + {B}_{3}^{T} {b}^{k} + {C}_{3}^{T} {c}^{k} ] \} \\ {b}^{k + 1} = {ρ}_{1} ( I - {prox}_{\frac{{λ}_{1}}{{ρ}_{1}} {φ}_{1}} ) ( \frac{{b}^{k}}{ρ} + {B}_{3} ( 2 {f}^{k + 1} - {f}^{k} ) ) \\ {c}^{k + 1} = {ρ}_{2} ( I - {prox}_{\frac{{λ}_{2}}{{ρ}_{2}} {φ}_{2}} ) ( \frac{{c}^{k}}{ρ} + {C}_{3} ( 2 {f}^{k + 1} - {f}^{k} ) )\end{matrix} ,$$

wherein the b denotes (or corresponds to) a first subgradient term, the c denotes (or corresponds to) a second subgradient term, the C₃ denotes (or corresponds to) a three dimensional second order gradient block matrix, the φ₁ denotes (or corresponds to) a first order isotropic total variation norm, the φ₂ denotes (or corresponds to) a second order isotropic total variation norm, the λ₁ denotes (or corresponds to) a first regularization parameter, the λ₂ denotes (or corresponds to) a second regularization parameter, the S denotes a (or corresponds to) preconditioner, the ρ₁ denotes (or corresponds to) a first algorithmic parameter, and the ρ₂ denotes (or corresponds to) a second algorithmic parameter. In some embodiments, the method may further comprise using the CPSI to evaluate the condition of the subject.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device.

FIG. 1B is a block diagram depicting a cloud computing environment comprising client device in communication with cloud service providers.

FIGS. 1C and 1D are block diagrams depicting embodiments of computing devices useful in connection with the methods and systems described herein.

FIG. 2 illustrates an example system for implementing the disclosed approach for generating and/or reconstructing a CPSI based on a plurality of planar scintigraphy images, according to potential embodiments.

FIG. 3 illustrates a flow diagram of an example process for generating and/or reconstructing a CPSI corrected for image artifacts, according to potential embodiments.

FIG. 4 illustrates an example numerical phantom for evaluating the systems and methods described herein, according to potential embodiments.

FIG. 5 illustrates example photo-peak energies from a decay chain for evaluating the systems and methods described herein, according to potential embodiments.

FIG. 6 illustrates example results of a performance evaluation of a system for generating and/or reconstructing a CPSI, according to potential embodiments. Reconstructed higher order total variation (HOTV) planar, post-filtered geometric mean, geometric mean, and A/P images are shown from left to right (1 of 200 realizations) at three count levels (12,500, 25,000, and 50,000 counts) with a 50% scatter fraction. The HOTV and post-filtered geometric mean images were optimized with respect to minimum mean squared error.

FIGS. 7A-7I illustrate example results of a performance evaluation of a system for generating and/or reconstructing a CPSI, according to potential embodiments. Recovered contrast/background variability trade-off plots from 200 realizations are shown at three count levels (12,500 [FIGS. 7A-7C], 25,000 [FIGS. 7D-7F], and 50,000 [FIGS. 7G-7I] counts) using a 50% scatter fraction. Each target (small [FIGS. 7A, 7D, 7G], medium [FIGS. 7B, 7E, 7H], and large [FIGS. 7C, 7E, 7I]) are compared at three contrast levels.

FIG. 8 illustrates example results of a performance evaluation of a system for generating and/or reconstructing a CPSI, according to potential embodiments. Estimated values of contrast-to-noise ratios (CNR) for a particular noise level are shown at four count levels (e.g., 6,250 (6.25k) counts, 12,500 (12.5k) counts, 25,000 (25k) counts, and 50,000 (50k) counts).

FIG. 9 depicts energy resolution of NaI and spectrum of photopeaks from the ²²⁵Ac decay chain, ²²⁵Ac decay chain (²²⁵Ac→[t_{1/2}=9:92d]²²¹Fr→[t_{1/2}=4.8 min]²¹⁷At→[t_{1/2}=33 μs]²¹³Bi→t_{1/2}=45.6 min]) with proposed energy windows to be used for imaging. Peaks, weighted with their respective branching ratios, are plotted according to the ratios found in the table of FIG. 5. The resolution of the NaI gamma camera is calibrated at 6.5% at 662 keV, with an inverse of the square root of E energy dependence. In certain embodiments, other isotopes (e.g., ²²⁷Th, ²²³Ra, ¹⁷⁷Lu, ¹³¹I, and/or other isotopes) can be used to evaluate the systems and methods described herein.

FIG. 10 illustrates example images with degradation and/or artifacts caused by one or more physical processes, according to potential embodiments.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes embodiments of systems and methods of the present technology for reconstructing and/or generating a corrected planar scintigraphy image (CPSI) corrected for (e.g., without or with reduced) image artifacts based on a plurality of planar scintigraphy images.

### A. Computing and Network Environment

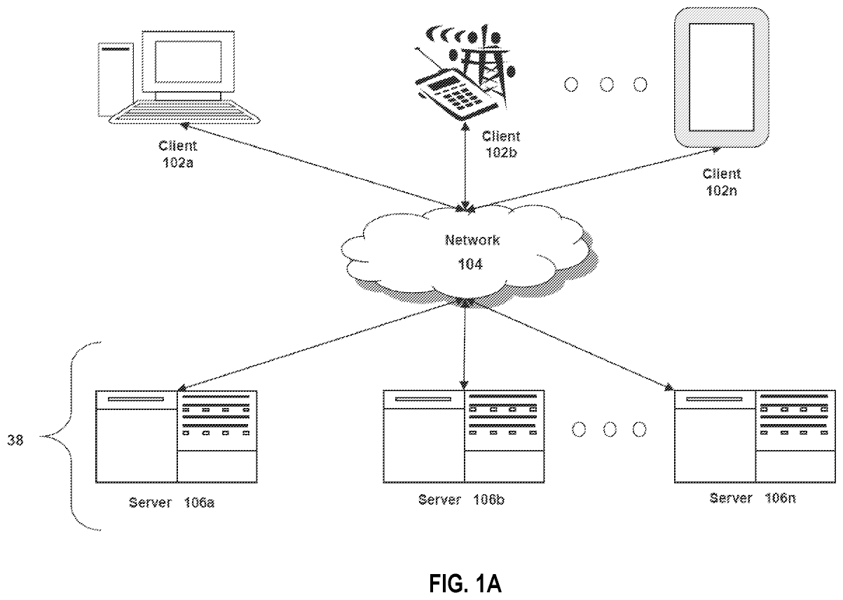

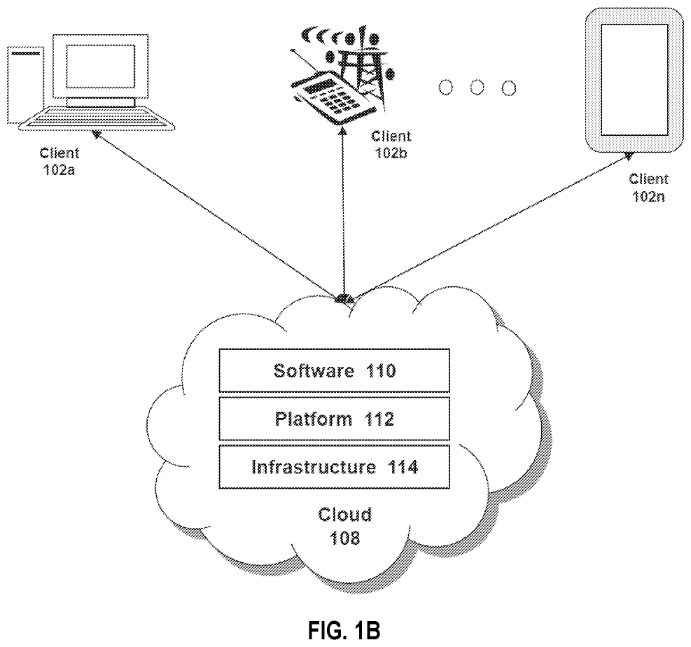

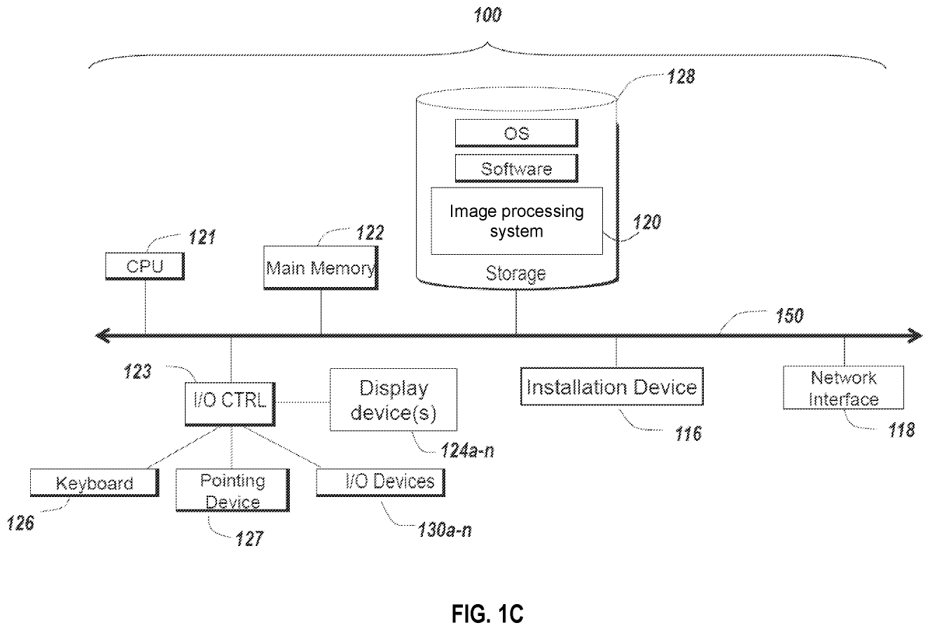

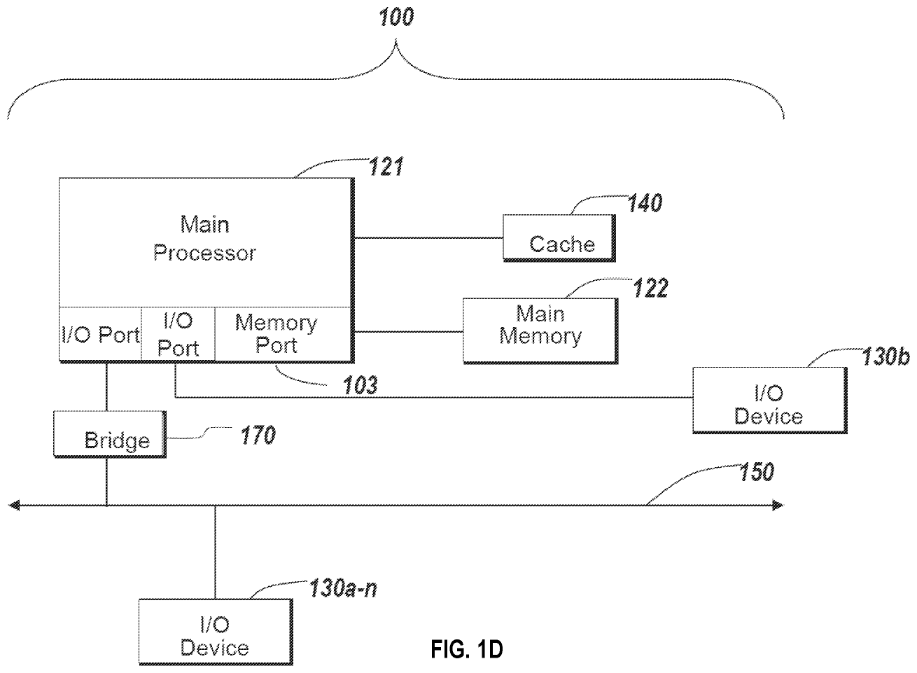

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous-one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of an image data processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the image processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, WINDOWS 8, and WINDOWS 10 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Generating a Corrected Planar Scintigraphy Image (CPSI)

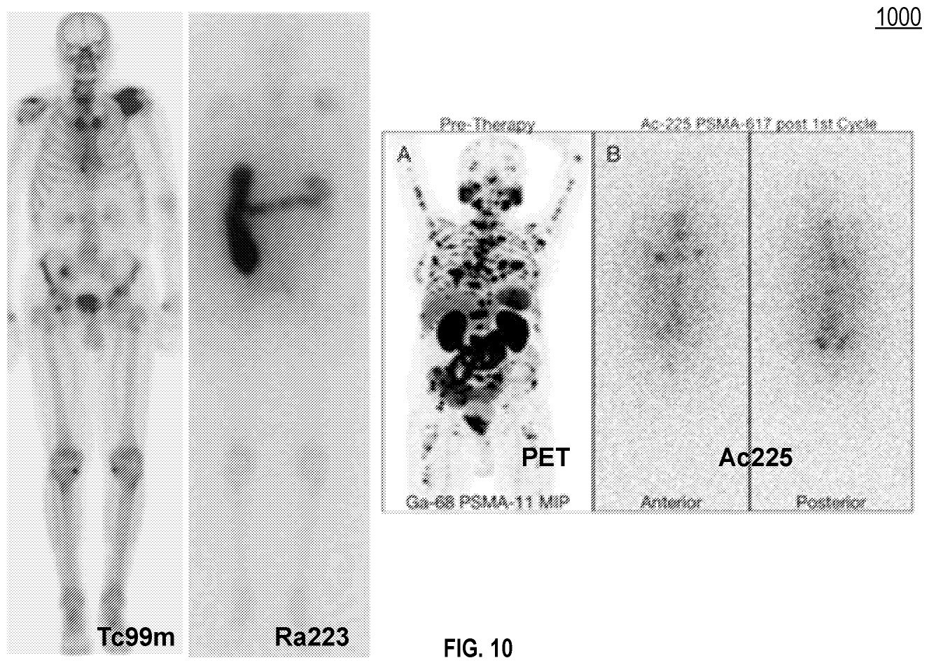

In medical imaging applications (e.g., planar scintigraphy imaging and/or other types of medical imaging), radiologists and/or physicians can view/analyze one or more medical images of a subject (e.g., anterior/posterior (A/P) image) for diagnostic and/or treatment purposes. Certain medical image(s), such as planar scintigraphy images, may describe and/or indicate an accumulated count distribution produced by collimated single photon emissions within said subject. Similar to other types of medical images (e.g., reconstructed 3D tomographic images), planar scintigraphy images may contain degradation and/or artifacts caused by one or more physical processes (such as attenuation, scatter, or collimator penetration). The image degradation/artifacts can obscure/obfuscate certain physiological/anatomical information within the planar scintigraphy images, thereby reducing the clinical value and/or usability of the medical images for answering clinical questions. FIG. 10, for example, depicts one or more example Tc99m/Ra223 and/or PET/Ac225 images with degradation and/or artifacts caused by one or more physical processes, wherein the degradation/artifacts obscure physiological/anatomical information within the example images.

In some imaging modalities, the use of certain radionuclides (such as radionuclides that emit high energy photons and/or have low counts) may cause an increased amount of imaging artifacts within the planar scintigraphy images (compared to using other types of radionuclides). For instance, radionuclides used in targeted alpha therapy (TAT) can have a low photon yield, long half-lives, and/or a plurality of photon energies (including high-energy photons), which due to radiation dose constraint, can limit the administered TAT activity (e.g., administered to the subject). Further, imaging times may not be sufficiently extended to increase the yield of imageable photons. As a result, planar scintigraphy images can be excessively noisy and contain imaging artifacts. In TAT, alpha emitting molecules can be used to target and/or treat cancerous cells/tissue within a subject. However, for the reasons discussed above, the quality of a TAT planar scintigraphy image may not allow for patient-specific prescriptions of administered TAT activity that optimize treatment of cancerous tissue (e.g., maximize the probability of cure), while minimizing a risk of toxicity to non-cancerous tissue. As such, improving the image quality of planar scintigraphy images (e.g., removing the image artifacts caused by physical processes) can enable image-guided optimization of TAT activity in a subject, thereby allowing for an assessment and/or evaluation of the therapeutic benefits of TAT.

A plurality of approaches have been applied to planar scintigraphy images in an attempt to improve the quality (e.g., removing the image artifacts) of said images, such as denoising by post-filtering via wavelet transformation, Fourier and wavelet transformations, contourlets, bandelets, complex ridgelets, curvelets, blind-deconvolution schemes, statistically adaptive methods, and/or Monte Carlo methods. However, said approaches fail to consider and/or include a physical/system model for planar scintigraphy, and as such, are unable to remove the image artifacts caused by one or more physical processes (e.g., attenuation, scatter, and/or collimator penetration). Furthermore, applying said approaches to the planar scintigraphy images can reduce the spatial resolution of the images.

Embodiments of the systems and methods presented herein include a novel approach for generating and/or reconstructing a CPSI corrected for said image artifacts (e.g., without reducing spatial resolution) by using a plurality of planar scintigraphy images, such as an anterior planar scintigraphy image and a posterior planar scintigraphy image. The disclosed approach is based on a physical model of a two-view SPECT system, in which an anterior planar scintigraphy image may correspond to a posterior planar scintigraphy image upon removal of the image artifacts from the planar scintigraphy images. In certain embodiments, a planar scintigraphy image reconstruction model (based on the two-view SPECT physical model) can be applied to the plurality of planar scintigraphy images to generate the CPSI. The planar scintigraphy image reconstruction model can be based on a novel regularized image reconstruction approach (e.g., based on sparse image representation), in which the two-view SPECT physical model can be used as a constraint for computing and/or estimating a total activity bio-distribution between a plurality of gamma ray detectors (e.g., detectors for generating the plurality of planar scintigraphy images).

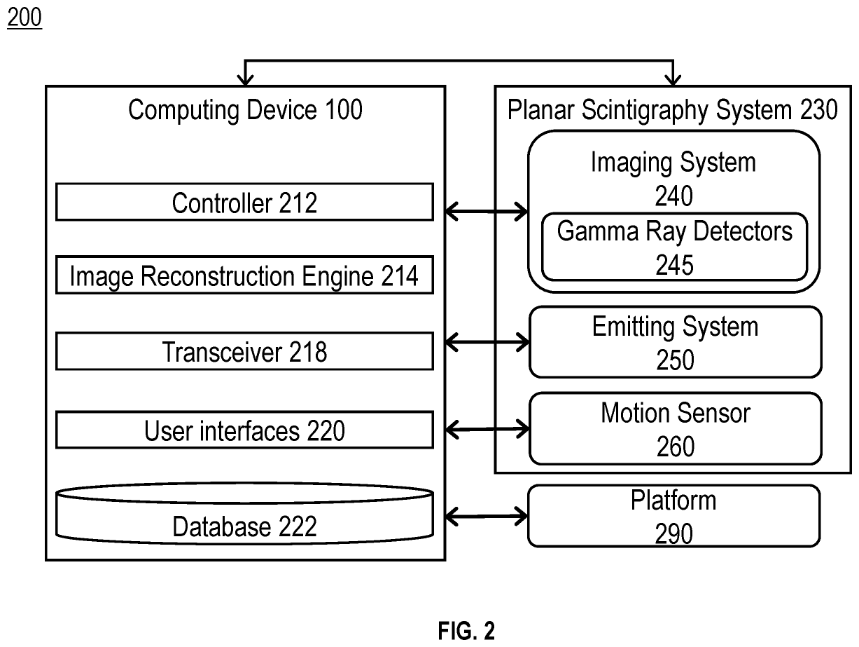

Referring to FIG. 2, in various embodiments, a system 200 may include a computing device 100 (or multiple computing devices, co-located or remote to each other), an imaging system 240 (which may include, e.g., a gamma scanner, or other imaging devices and sensors, such as gamma ray detectors/cameras 245), an emitting system 250 (which may include, e.g., a gamma ray emission system and/or one or more other devices), and and/or a motion sensor 160. In various implementations, the imaging system 240, the emitting system 250, and/or the motion sensor 260 (e.g., to detect motion by a subject) may be integrated into one planar scintigraphy system 230. In certain implementations, computing device 100 (or components thereof) may be integrated with one or more of the planar scintigraphy system 230, imaging system 240, emitting system 250, and/or motion sensor 260. The planar scintigraphy system 230, imaging system 240, emitting system 250, and/or motion sensor 260 may be directed to a platform 290 on which a patient or subject can be situated (so as to image the subject, apply a treatment or therapy to the subject, and/or detect motion by the subject). In various embodiments, the platform 290 may be movable (e.g., using any combination of motors, magnets, etc.) to allow for positioning and repositioning of subjects (such as micro-adjustments due to subject motion).

The computing device 100 (or multiple computing devices) may be used to control and/or receive signals acquired via imaging system 240, emitting system 250, and/or motion sensor 260 directly. In certain implementations, computing system 100 may be used to control and/or receive signals acquired via the planar scintigraphy system 230. For instance, the computing device 100 may receive and/or obtain a plurality of scintigraphy images of a subject (and/or other signals) from the imaging system 240, wherein the gamma ray detectors 245 of the imaging system 240 can be used to obtain/capture the planar scintigraphy images. The computing device 100 may include one or more processors and one or more volatile and non-volatile memories for storing computing code and data that are captured, acquired, recorded, and/or generated (e.g., captured, acquired, recorded and/or generated by the computing device 100 and/or the planar scintigraphy system 230). The computing device 100 may include a controller 212 that is configured to exchange control signals with the planar scintigraphy system 230, imaging system 240, emitting system 250, motion sensor 260, and/or platform 290, allowing the computing device 100 to be used to control the capture of images (e.g., planar scintigraphy images and/or other types of images) and/or signals via the sensors (e.g., gamma ray detectors 245) thereof, and position or reposition the subject. The computing device 100 may also include an image reconstruction engine 214 configured to perform the computations and analyses discussed herein with respect to generating a CPSI. For example, the image reconstruction engine 214 can apply a planar scintigraphy image reconstruction model to a plurality of planar scintigraphy images (e.g., captured, generated, or otherwise obtained via the imaging system 240).

A transceiver 218 allows the computing device 100 to exchange readings, control commands, and/or other data with the planar scintigraphy system 230, the imaging system 240, the emitting system 250, the motion sensor 260, and/or the platform 290 wirelessly or via wires. One or more user interfaces 220 (e.g., I/O devices 130) can allow the computing device 100 to receive user inputs (e.g., via a keyboard, touchscreen, microphone, camera, etc.) and provide outputs (e.g., via a display screen, audio speakers, etc.). For instance, the one or more user interfaces 220 can allow the computing device 100 to present the CPSI for evaluation of a condition of the subject (e.g., via a display 124 of the planar scintigraphy system 230 and/or the computing device 100). The computing device 100 may additionally include one or more databases 222 for storing, for example, signals acquired via one or more sensors, signatures, etc. For example, the database(s) 222 may store and/or maintain the planar scintigraphy images and/or the generated CPSI. In some implementations, database 222 (or portions thereof) may alternatively or additionally be part of another computing device that is co-located or remote and in communication with computing device 100, planar scintigraphy system 230, imaging system 240, emitting system 250, motion sensor 260, and/or platform 290.

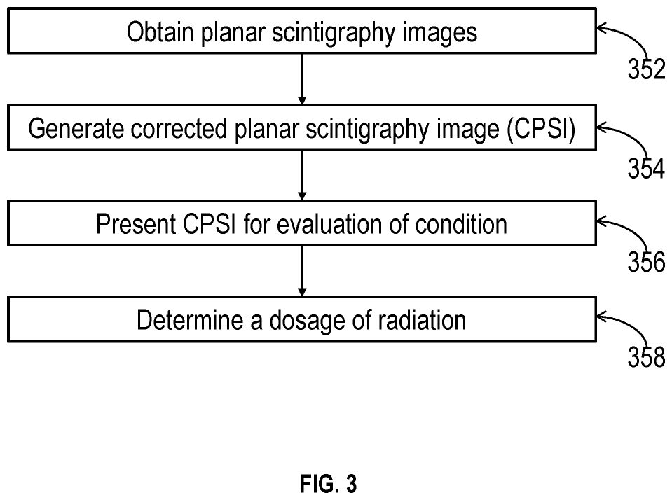

Referring to FIG. 3, depicted is a flow diagram of an embodiment of a method for generating and/or reconstructing a CPSI (or other medical images) corrected for (e.g., without or with reduced) image artifacts (e.g., artifacts caused by gamma ray attenuation, gamma ray collimator penetration, gamma ray scatter, and/or other processes) by using (or based on) a plurality of planar scintigraphy images. The functionalities of the method may be implemented using, or performed by, the components detailed herein in connection with FIGS. 1A-1D and 2. In some embodiments, process 350 can be performed by a client 102 or a server 106. In some embodiments, process 350 can be performed by other entities, such as a computing device 100 and/or a planar scintigraphy system 230 (as discussed in FIGS. 1C-1D and 2). In some embodiments, process 350 may include more, fewer, or different steps than shown in FIG. 3.

In brief overview, process 350 can include obtaining a plurality of planar scintigraphy images (352). The process 350 may include generating a corrected planar scintigraphy image (CPSI) (354). The process 350 may include presenting the generated CPSI for evaluation of a condition of a subject (356). The process 350 may include determining a dosage of radiation according to the CPSI (358).

Referring now to operation (352), and in some embodiments, a computing system (e.g., a client 102, a server 106, and/or a computing device 100) may obtain, receive, and/or acquire a plurality of planar scintigraphy images (or other medical images) of a subject. For instance, the computing system may obtain the plurality of planar scintigraphy images by generating and/or capturing said images using a plurality of gamma ray detectors (e.g., gamma ray cameras). In one example, the computing system may obtain the plurality of planar scintigraphy images by receiving (e.g., according to one or more signals) said images from another computing system (e.g., a medical imaging system) and/or computing device 100. The plurality of planar scintigraphy images may contain and/or include image artifacts caused by one or more physical processes. For instance, the plurality of planar scintigraphy images may contain image artifacts caused by gamma ray attenuation, gamma ray collimator penetration, gamma ray scatter, and/or other physical processes. In some embodiments, the plurality of planar scintigraphy images may include/comprise an anterior planar scintigraphy image and/or a posterior planar scintigraphy image. In certain embodiments, the anterior planar scintigraphy image may include or correspond to the posterior planar scintigraphy image upon removal of the image artifacts and/or image noise from said images. Certain processes (such as determining a geometric mean of the plurality of planar scintigraphy images) may remove (e.g., from the planar scintigraphy images) a portion of the image artifacts caused by gamma ray attenuation, but are unable to remove image artifacts caused by gamma ray collimator penetration, gamma ray scatter, and/or other physical processes.

Referring now to operation (354), and in some embodiments, the computing system may generate a CPSI (e.g., based on the plurality of planar scintigraphy images) corrected for (e.g., without) the image artifacts of the plurality of planar scintigraphy images. For instance, the computing system may generate said CPSI by applying a planar scintigraphy image reconstruction model to the plurality of planar scintigraphy images (e.g., processing the plurality of planar scintigraphy images with the planar scintigraphy image reconstruction model). The planar scintigraphy image reconstruction model may comprise a non-negativity constraint (e.g., values equal to or greater than zero) and/or other types of constraints. The planar scintigraphy image reconstruction model can be based on a regularization term and/or a fidelity term. The fidelity term (e.g., a divergence norm and/or other types of norms or semi-norms, such as a L1-norm and/or a KL-norm) can measure, determine, and/or quantify how a first probability distribution (e.g., a probability distribution associated to the plurality of planar scintigraphy images) is different from a second probability distribution (e.g., a probability distribution associated to a model of a two-view SPECT system). In some embodiments, the regularization term may correspond to a total variation (TV) regularization (and/or other types of regularization, such as tight framelets based on a redundant multiscale discrete cosine transform and/or deep learning approaches) for controlling noise (e.g., count noise). In certain examples, the regularization term may include or correspond to other types of regularization for controlling noise, such as deep learning, sparse representation, quadratic, and/or other types.

In some embodiments, the planar scintigraphy image reconstruction model may comprise a minimization operation (e.g., a minimization of a mode) based on the regularization term, the fidelity term, and/or the non-negativity constraint. For instance, the planar scintigraphy image reconstruction model may comprise a minimization of a sum (or other operation(s)) of the regularization term, the fidelity term, and/or the non-negativity constraint (e.g., to estimate and/or generate the CPSI). In some embodiments, the minimization operation can be based on a divergence norm (e.g., a Kullback-Leibler (KL) divergence norm and/or other types of norms) and/or a regularization parameter (λ). For example, the minimization operation can include a minimization of a KL divergence norm (or other norms). In certain embodiments, the regularization parameter may determine a weight of the regularization term within the planar scintigraphy image reconstruction model.

In some embodiments, the planar scintigraphy image reconstruction model may be based on (or correspond to) a two-view single photon emission computed tomography (SPECT) physical model. In one example, the process 300 may include applying or solving the SPECT physical problem/model to generate the CPSI. The two-view SPECT physical model may comprise a conjugate pair of detectors/images, such as an anterior view and a posterior view (e.g., a conjugate anterior/posterior (A/P) pair of images/detectors, a right/left pair of images/detectors, and/or an angle/angle+180 degrees pair of images detectors). In certain embodiments, generating the CPSI may comprise estimating conjugate views, such as an A/P projection of activity bio-distribution (e.g., a bio-distribution of a molecular target, such as a bio-distribution of TAT tracers) using the two-view SPECT physical model as a constraint. In some embodiments, the two-view SPECT physical model can be determined (e.g., determined by the computing system) according to ∫_{J}_{³}K(x; y)ƒ(y)dy=g (x), x∈J³, wherein x:=(x₁, x₂, x₃) and/or y:=(y₁, y₂, y₃). The g(x) can describe or correspond to the plurality of planar scintigraphy images. The K(x; y) may describe or correspond to a kernel of a region J:=[α, b]. The kernel K(x; y) can be determined (e.g., by the computing system) according to K(x; y):=δ(x₁−y₁, x₂−y₂)e^{-∫}^{_{y3}}^{^{x3}}^{μ(y1,y2,t)dt} and/or other kernels that include accurate physical models (e.g., physical models that include resolution loss, collimator penetration, and/or scatter estimation). The μ(y), y∈J³ can describe or correspond to an attenuation map derived from computed tomography (CT). The kernel K(x; y) can be determined and/or defined under the assumption of an ideal collimator and detectors, and/or a known attenuation map μ(y). In some embodiments, the kernel K(x; y) may include and/or consider an additive term (e.g., s (x)) describing scatter. The additive term can be estimated independently/separately.

In certain embodiments, applying the planar scintigraphy image reconstruction model may comprise determining ƒ̅(y₁, y₂)=∫_{J} ƒ(y₁, y₂, y₃)dy₃ subject to:

$${min}_{f} \{ {‖ {\int }_{{J}^{3}} K ( \cdot ; y ) f ( y ) dy - g ( \cdot ) ‖}_{KL} + λ {‖ f ‖}_{TV ( {J}^{3} )} + {ι}_{+} ( f ) \} .$$

The ƒ̅(y₁, y₂) can describe the CPSI (e.g., the average/projected activity distribution in y₃). The ∥∫_{J}_{³} K(·; y)ƒ(y)dy−g(·)∥_{KL} can denote (or describe) a divergence norm (e.g., a KL divergence norm and/or other types of norms). In some embodiments, the divergence norm may include or correspond to the fidelity term. In certain embodiments, the fidelity term may include or correspond to other types of norms, such as an L1-norm, a KL-norm, and/or other types of norms. In certain embodiments, the λ∥ƒ∥_{TV(J}_{³}₎ can describe a total variation regularization for controlling noise. As such, the λ∥ƒ∥_{TV(J}_{³}₎ may include or correspond to the regularization term. In some embodiments, the regularization term may include or correspond to other types of regularization approaches, such as quadratic, sparse representation, and deep learning. In some embodiments, the l₊(ƒ) may include or correspond to an indicator function (or other types of functions). The indicator function may impose the non-negativity constraint (e.g., imposing a non-negativity constraint on ƒ) of the planar scintigraphy reconstruction model. In some embodiments, and as described above, the kernel K(x; y) can be determined (e.g., by the computing system) according to K(x; y): =δ(x₁−y₁, x₂−y₂)e^{-∫}^{_{y3}}^{^{x3}}^{μ(y1,y2,t)dt} and/or other kernels that include accurate physical models (e.g., models that include resolution loss, collimator penetration, and/or scatter estimation). As such, applying the planar scintigraphy image reconstruction model may comprise determining \hat{ƒ}(y₁, y₂)=∫_{J} ƒ(y₁, y₂, y₃) dy₃ subject to: min {∥∫_{J}e^{-∫}^{_{y3}}^{^{x3}}^{μ(y1,y2,t)dt} ƒ(y₁, y₂, y₃)dy₃−g(y₁, y₂)∥_{KL}+λ∥ƒ∥_{TV(J}_{³}₎+l₊(ƒ)}. The ∥∫_{J}e^{-∫}^{_{y3}}^{^{x3}}^{μ(y1,y2,t)dt} ƒ(y₁, y₂, y₃)dy₃−g (y₁, y₂)∥_{KL} can denote (or describe) a divergence norm (e.g., a KL divergence norm and/or other types of norms). In some embodiments, the divergence norm may include or correspond to the fidelity term. In certain embodiments, the fidelity term may include or correspond to other types of norms, such as an L1-norm, a KL-norm, and/or other types of norms.

In some embodiments, the process 300 may include discretizing (e.g., by the computing system) the planar scintigraphy image reconstruction model. The planar scintigraphy image reconstruction model can be discretized according to ƒ̅=Σ_{y}_{₃}ƒ(y), subject to:

$${min}_{f} \{ {‖ Kf - g ‖}_{KL} + λ {‖ {B}_{3} f ‖}_{1} + {ι}_{+} ( f ) \} .$$

The KL-divergence norm can be given by ∥Kf−g|_{KL}=Kf−g log(Kƒ). The B₃ may include or correspond to a three dimensional first order gradient block matrix. In certain embodiments, the discretized planar scintigraphy image reconstruction model can be applied or solved via a fixed-point approach and/or other types of approaches, such as ADMM and/or Chambolle-Pock. For instance, the discretized planar scintigraphy image reconstruction model can be applied or solved using a fixed-point algorithm with higher order total variation regularization (HOTV) and/or other types of regularization according to:

$$\{ \begin{matrix}{f}^{k + 1} = {prox}_{{ι}_{+}} \{ {f}^{k} - S [ {K}^{T} ( 1 - \frac{g}{{Kf}^{k}} ) + {B}_{3}^{T} {b}^{k} + {C}_{3}^{T} {c}^{k} ] \} \\ {b}^{k + 1} = {ρ}_{1} ( I - {prox}_{\frac{{λ}_{1}}{{ρ}_{1}} {φ}_{1}} ) ( \frac{{b}^{k}}{ρ} + {B}_{3} ( 2 {f}^{k + 1} - {f}^{k} ) ) \\ {c}^{k + 1} = {ρ}_{2} ( I - {prox}_{\frac{{λ}_{2}}{{ρ}_{2}} {φ}_{2}} ) ( \frac{{c}^{k}}{ρ} + {C}_{3} ( 2 {f}^{k + 1} - {f}^{k} ) )\end{matrix} .$$

In certain embodiments, the b may include or correspond to a first subgradient term, while the c may include or correspond to a second subgradient term. The C₃ may describe, include or correspond to a three dimensional second order gradient block matrix. The φ₁ may include or correspond to a first order isotropic total variation norm, while the φ₂ may include or correspond to a second order isotropic total variation norm. In some embodiments, the λ₁ may include or correspond to a first regularization parameter (e.g., a weight of a regularization term), while the λ₂ may include or correspond to a second regularization parameter. In certain embodiments, the S may include or correspond to a preconditioner. The ρ₁ may include or correspond to a first algorithmic parameter, while the ρ₂ may include or correspond to a second algorithmic parameter. The proxₗ₊ may include or correspond to a proximity operator of the indicator function (e.g., for the non-negativity constraint). In some embodiments, the

$${prox}_{\frac{λ}{ρ} φ}$$

may include or correspond to a proximity operator of an isotropic total variation norm. In

$${\overline{f}}_{i , j} = \frac{1}{Δ k} {\sum }_{k} {\hat{f}}_{i , j , k} ,$$

the \hat{ƒ} may include or correspond to a converged solution (e.g., \hat{ƒ}≈ƒ^{k=∞}) to the planar scintigraphy image reconstruction model, wherein the Δk describes a voxel spacing. Although the recovery of \hat{ƒ} represents an ill-posed problem, the mean information in y₁/y₂ can be recovered by summing along y₃.

In certain embodiments, the Moreau envelope function for a convex function ψ: Rⁿ→R can be defined/determined by (or according to)

$${prox}_{ψ} ( x ) = arg {min}_{u} \{ \frac{1}{2} {‖ u - x ‖}_{2}^{2} + ψ ( u ) : u ∈ {R}^{n} \} .$$

For B₃ and C₃, N=\sqrt{d}. In some embodiments, I_{N} may include or correspond to a N×N identity matrix, while D can describe a N×N backward difference matrix such that D_{j,j}=1 and D_{j,j-1}=−1 for j=2,1, . . . N. As such, the other entries of D may include or correspond to zero. Through a matrix Kronecker product ⊗, B₃∈R^{3N} and C₃∈R^{9N} can be determined and/or described according to:

$${B}_{3} := [ \begin{matrix}{I}_{N} & ⊗ & {I}_{N} & ⊗ & D \\ {I}_{N} & ⊗ & D & ⊗ & {I}_{N} \\ D & ⊗ & {I}_{N} & ⊗ & {I}_{N}\end{matrix} ] , \\ {C}_{3} := [ \begin{matrix}{I}_{N} & ⊗ & {I}_{N} & ⊗ & ( - {D}^{T} ) D \\ {I}_{N} & ⊗ & ( - {D}^{T} ) & ⊗ & D \\ ( - {D}^{T} ) & ⊗ & {I}_{N} & ⊗ & D \\ {I}_{N} & ⊗ & D & ⊗ & ( - {D}^{T} ) \\ {I}_{N} & ⊗ & ( - {D}^{T} ) D & ⊗ & {I}_{N} \\ ( - {D}^{T} ) & ⊗ & D & ⊗ & {I}_{N} \\ D & ⊗ & {I}_{N} & ⊗ & ( - {D}^{T} ) \\ D & ⊗ & ( - {D}^{T} ) & ⊗ & {I}_{N} \\ ( - {D}^{T} ) D & ⊗ & {I}_{N} & ⊗ & {I}_{N}\end{matrix} ]$$

In some embodiments, the first order isotropic total variation norm φ₁ can be defined/determined by (or according to)

$${φ}_{1} ( x ) = {\sum }_{i = 1}^{d} \sqrt{{\sum }_{j = 0}^{2} {x}_{i + jd}^{2}} .$$

In certain embodiments, the second order isotropic total variation norm φ₂ can be defined/determined by (or according to)

$${φ}_{2} ( y ) = {\sum }_{i = 1}^{d} \sqrt{{\sum }_{j = 0}^{8} {y}_{i + jd}^{2}} .$$

In certain embodiments, the preconditioner by (or according to) S can be defined by

$$S = \frac{{f}^{k}}{{K}^{T} 1} .$$

The algorithmic parameters ρ_{1,2} can be defined by

$${ρ}_{1 , 2} = \frac{1}{2 {‖ {B}_{3} , {C}_{3} ‖}_{2}^{2} {‖ S ‖}_{∞}} ,$$

wherein

$${‖ {B}_{3} ‖}_{2}^{2} = 12 and {‖ {C}_{3} ‖}_{2}^{2} = 144.$$

The regularization parameters λ_{1,2} can be determined according to a mean squared error approach (and/or other approaches).

Referring now to operation (356), and in some embodiments, the computing system may present, display, or otherwise provide the CPSI for evaluation (e.g., evaluation by a clinician) of a condition of the subject. In one example, a clinician may evaluate the CPSI and/or other information to determine a medical condition of the subject. The computing system may present the CPSI by transmitting, sending, and/or communicating the CPSI to a computing device 100 and/or displaying the CPSI on a display screen (e.g., one or more display devices 124a-124n).

Referring now to operation (358), and in some embodiments, the process 300 can include determining a dosage of radiation administered to the subject (e.g., to treat a medical condition) according to the generated CPSI (e.g., to determine a therapeutic benefit and/or a risk of toxicity). For instance, a dose of administered TAT activity (e.g., administered to the subject) can be determined according to the CPSI, wherein the determined dose of administered TAT activity can minimize/reduce a risk of toxicity to non-cancerous tissue while optimizing treatment for cancerous tissue. As such, the generated CPSI (with an improved image quality compared to the plurality of planar scintigraphy images) can enable an assessment and/or evaluation of the therapeutic benefits of TAT for treating a condition of the subject. In some embodiments, the generated CPSI can be used to evaluate a condition of the subject.

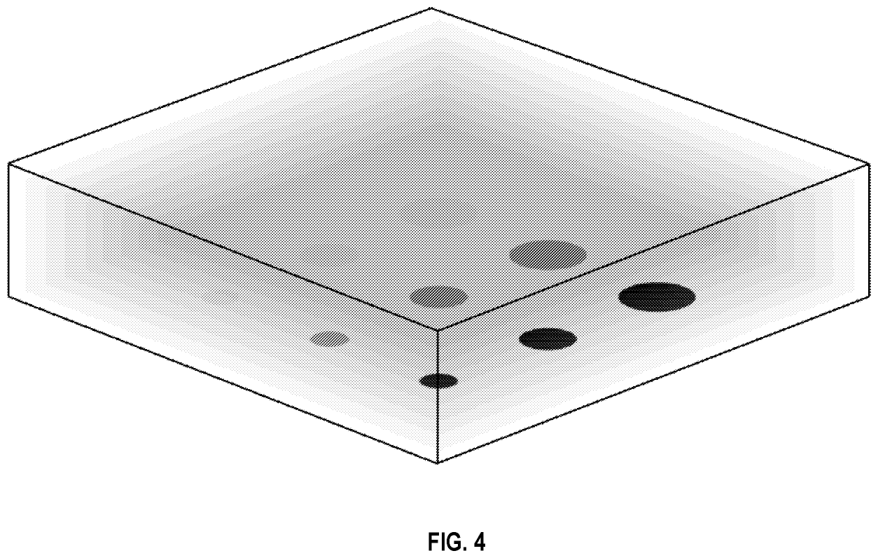

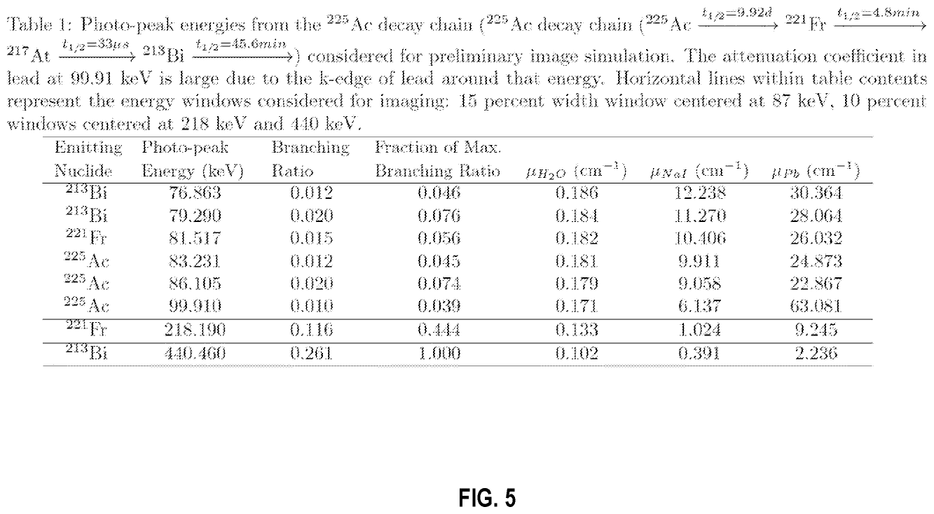

The performance of the systems and methods described herein can be evaluated according to (or by using) a numerical phantom with a 3×3 contrast detail object in a uniform background. The numerical phantom, as shown in FIG. 4, can be a 64×64×16 cm³ box with a grid of discs (e.g., discs with a thickness of 1 cm). The radii of the discs may include or correspond to 2 cm, 3 cm, and/or 4 cm. The intensity of the discs may include or correspond to 1.75, 2.125, and/or 2.5 times the A/P background counts. The discs can be inserted at a depth of 2 cm (or other depths) from a posterior ray detector. An Actinium-225 source can be used for the evaluation, wherein a plurality of short-lived daughter products of Actinium-225 (e.g., Francium-221, Astatine-217, and/or Bismuth-213) can be included. In certain embodiments, eight photon emissions can be taken into account (e.g., emissions with 1% or larger branching ratios), as shown in table 500 of FIG. 5. In some embodiments, a 50% (or other percentages) scatter background may be used for evaluating a performance of a system for generating a CPSI. The CPSI can be reconstructed/generated using 12500, 25000, and 50000 total counts with a HOTV penalty.

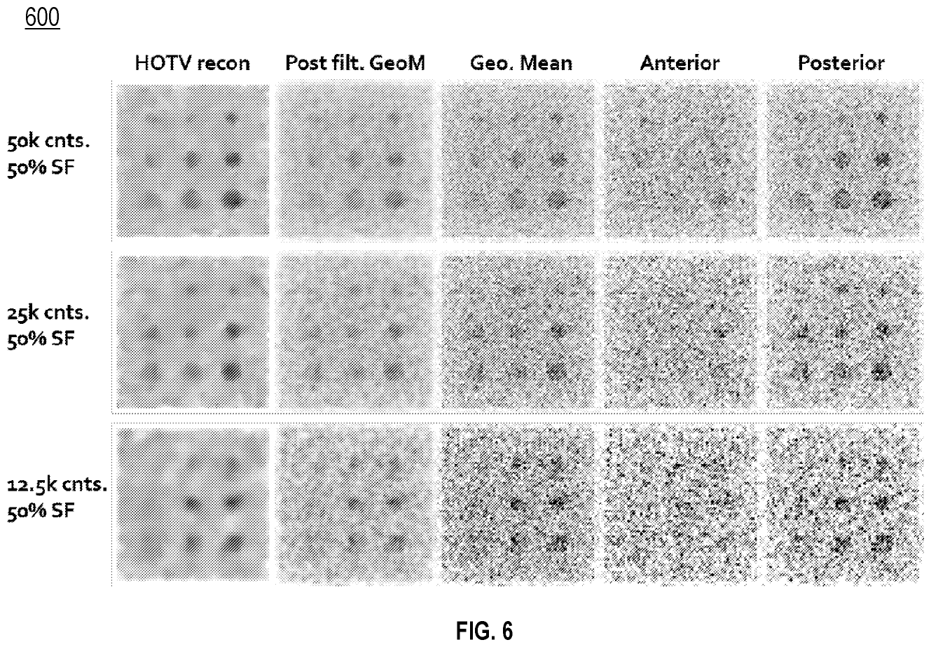

Referring to FIGS. 6, 7A-7I, and 8, depicted are example results of a performance evaluation of a system (e.g., a computing system) for generating and/or reconstructing a CPSI based on a plurality of planar scintigraphy images (e.g., anterior planar scintigraphy image and/or posterior planar scintigraphy image). FIG. 6 illustrates a reconstructed/generated CPSI (e.g., HOTV recon), a post-filtered geometric mean of the plurality of planar scintigraphy images (e.g., Post filt. GeoM), a geometric mean of the plurality of planar scintigraphy images (e.g., Geo. Mean), and a plurality of planar scintigraphy images (e.g., Anterior and Posterior) at three count levels (e.g., 12,500, 25,000, and 50,000 counts) with a 50% scatter function. The systems and methods presented herein can be used to generate the CPSI (e.g., HOTV recon). As shown in FIG. 6, the generated CPSI (e.g., generated based on the planar scintigraphy images) can have a higher/improved smoothness compared to a smoothness of a post-filtered geometric mean and/or a geometric mean of the plurality of planar scintigraphy images, while preserving/maintaining lesion conspicuity.

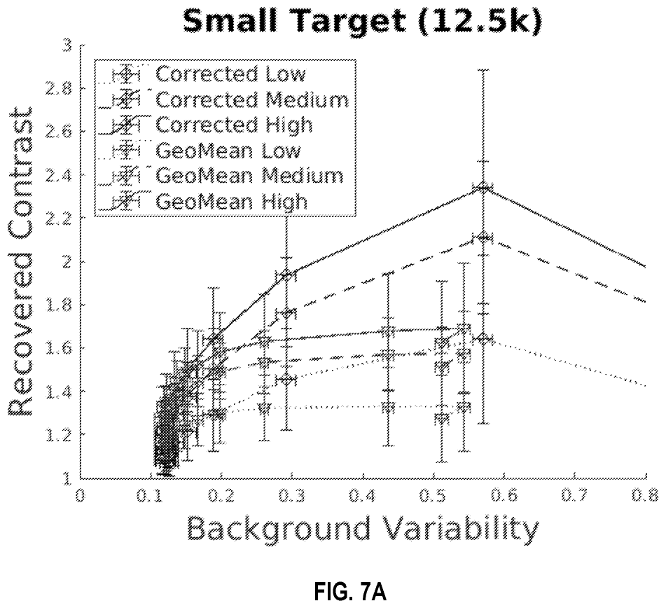

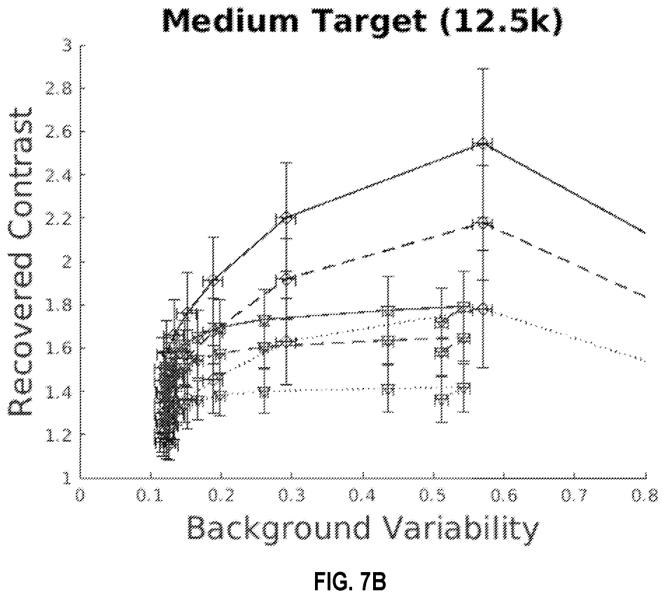

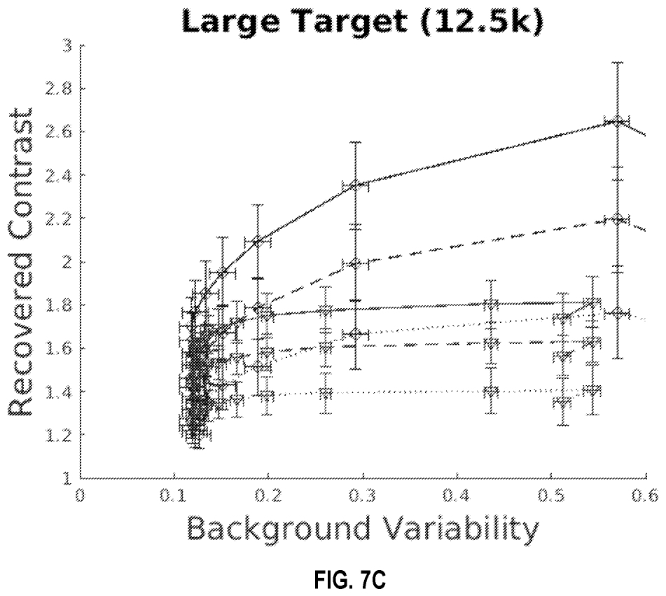

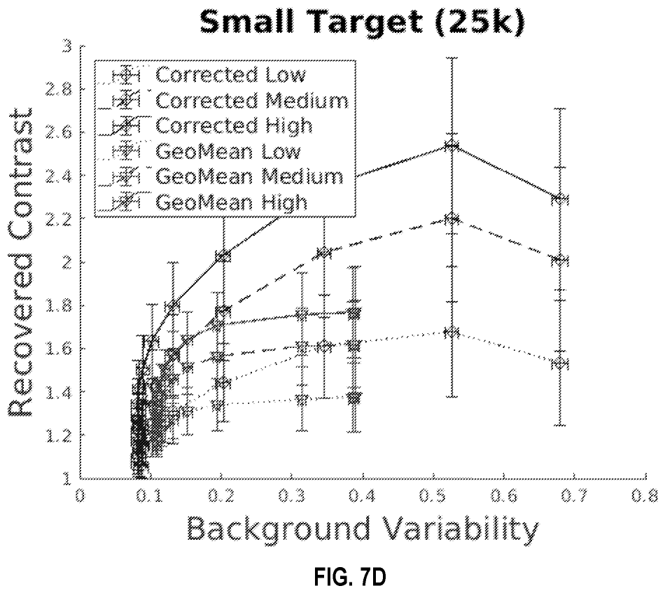

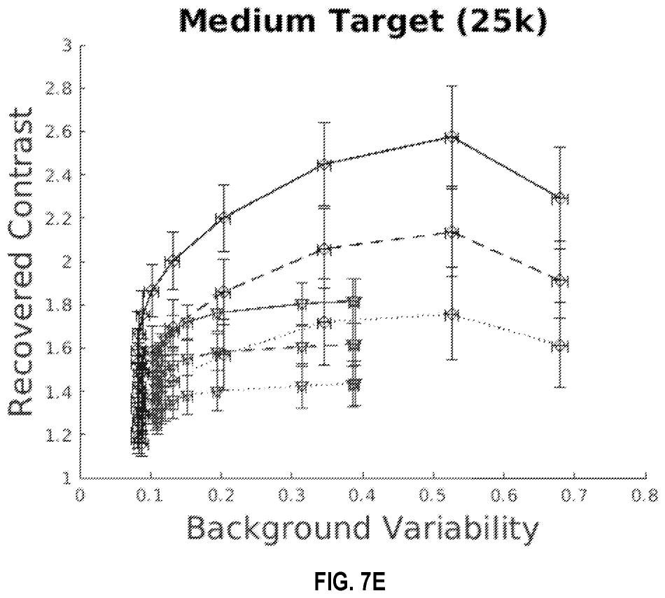

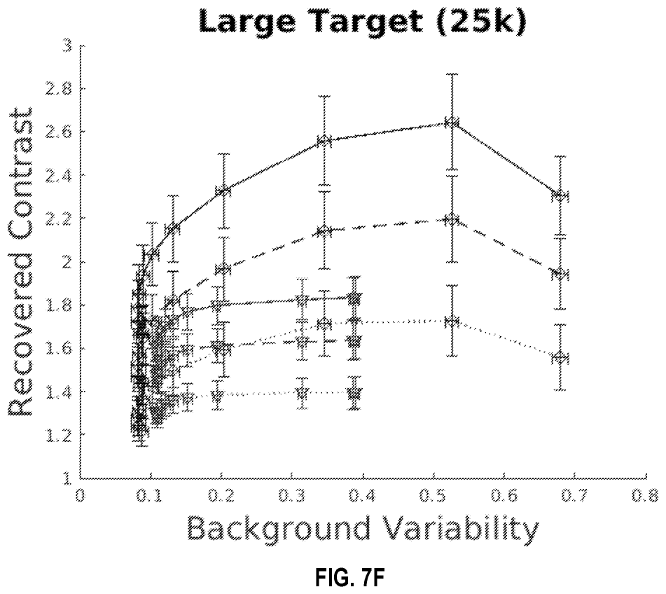

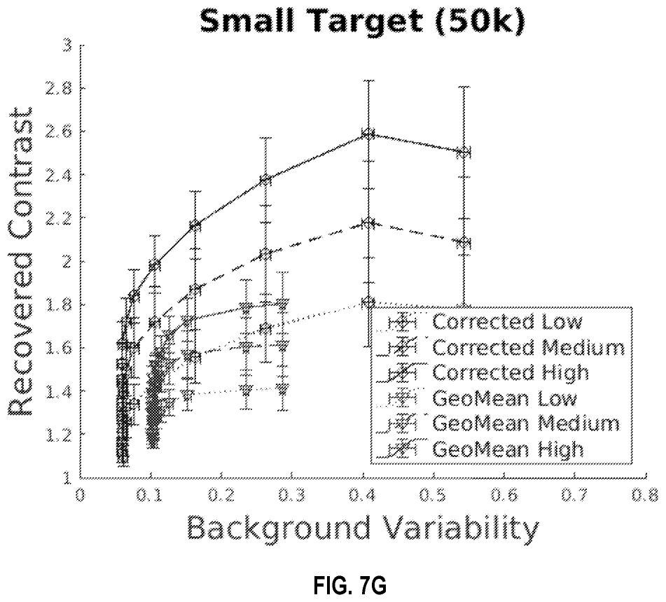

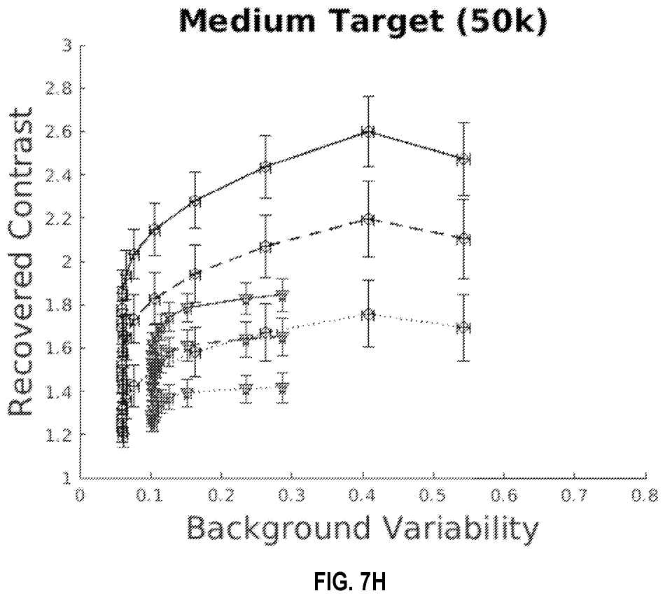

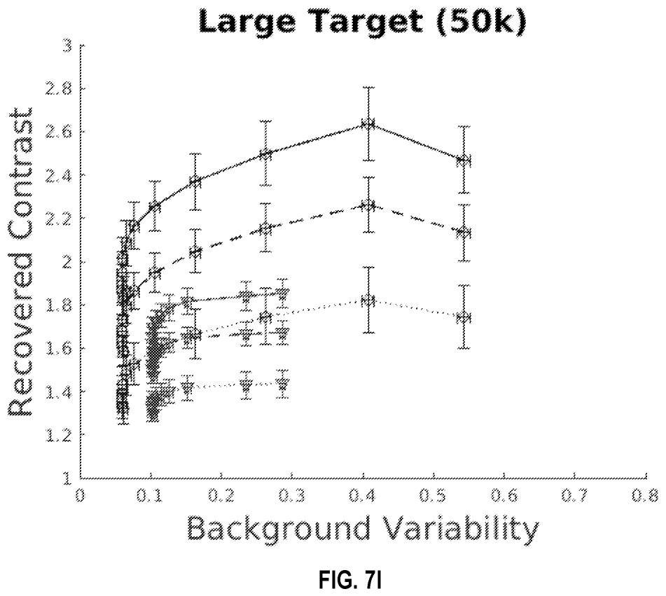

FIGS. 7A-7I depict a plurality of plots illustrating a recovered contrast versus background variability trade-off from 200 realizations at three count levels (e.g., 12,500, 25,000, and 50,000 counts) with a 50% scatter function. As such, FIGS. 7A-7I illustrate an example performance of the system for generating/reconstructing a CPSI across a range of values of a regularization parameter. For instance, FIGS. 7A-7I depict the tradeoff between a recovered contrast and a background variability (e.g., smoothness) as a function of a denoising strength at three contrast ratios. The denoising strength may include or correspond to the magnitude of the regularization parameter and the full width half maximum of a Gaussian post-filter for the corrected and geometric mean images, respectively. As seen in FIGS. 7A-7I, for each particular target at each particular contrast, the systems and methods described herein result in a higher contrast for a similar amount of noise when compared to the post-filtered geometric mean images. As shown in FIGS. 6 and 7A-7I, the proposed system for reconstructing/generating the CPSI outperforms conventional approaches (e.g., applying a geometric mean to planar scintigraphy images) for enhancing (e.g., improving the image quality of) planar scintigraphy images.

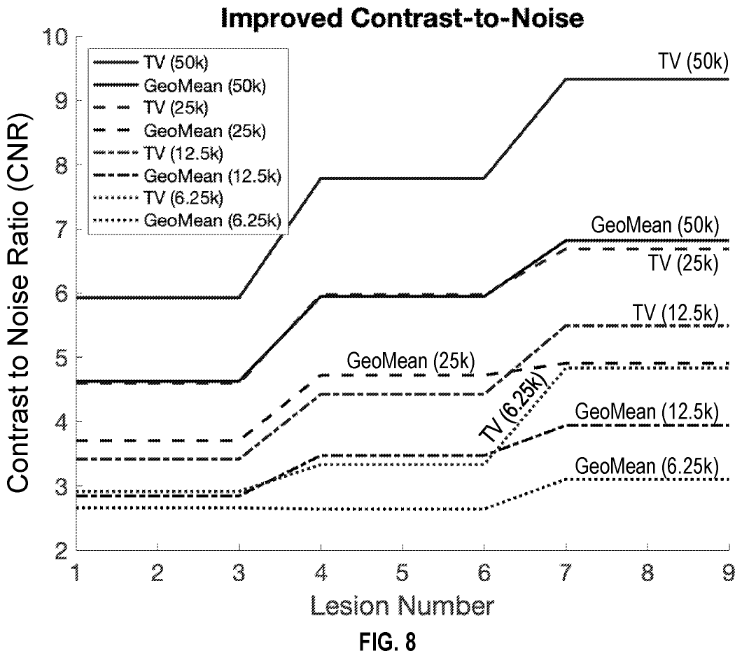

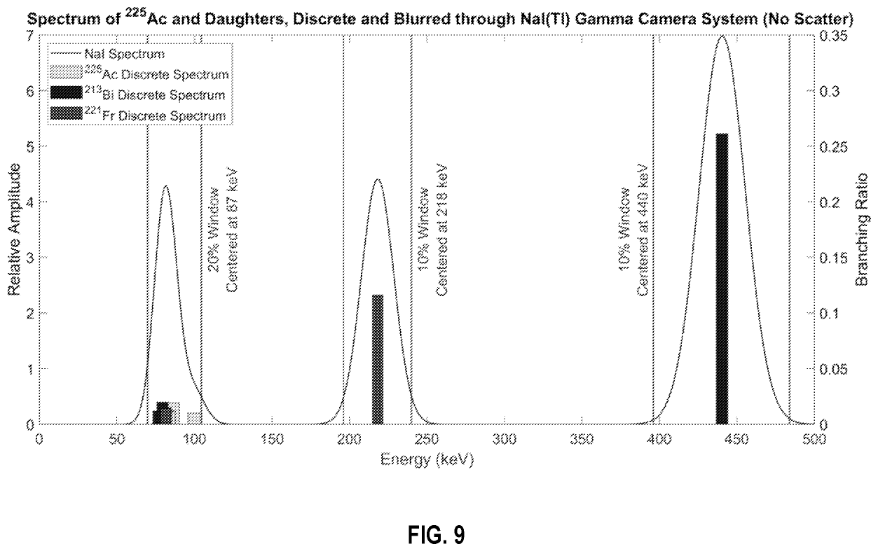

FIG. 8 illustrates example values of CNRs for a particular noise level at a plurality of count levels (e.g., 6.25k, 12.5k, 25k, and/or 50k). As show in FIG. 8, the systems and methods described herein can outperform a geometric mean approach (e.g., performing a geometric mean operation using the plurality of scintigraphy images) for generating a CPSI. For instance, the CNR(s) of said systems and methods can exceed the CNR(s) of the geometric mean approach by up to a factor of 4. In certain embodiments, the CNR of a particular lesion (from a total of nine lesions) of a contrast detail phantom (for example, the phantom illustrated in FIG. 4) were compared. The largest performance improvements (e.g., between the systems/methods described herein and the geometric mean approach) can be observed in the lower count images (e.g., lower of 6.25k, 12.5k, 25k, and/or 50k counts).

### Example Embodiments

The following embodiments of the systems and methods discussed herein are demonstrative of features of various alternative example embodiments.

Currently, there is no conventional curative approach for patients with advanced-stage metastatic prostate cancer. Happily, the recent remarkable success of the alpha-emitting radiopharmaceutical (AER) ²²⁵Ac-PSMA-617 in the treatment of metastatic castration-resistant prostate cancer (mCRPC) has revealed the striking clinical potential of Targeted Alpha Therapy (TAT).

TAT has been developing rapidly and has been applied as a treatment for many advanced-stage cancers in addition to mCRPC including leukemia, melanoma, bladder cancer, glioma, and neuroendocrine tumors. The two main benefits provided by alpha emitters are: (i) highly selective targeting of cancer cells while avoiding off-target cytotoxicity, due to the short α-particle ranges in tissue (<100 μm, i.e., a few cell diameters); and (ii) enhanced cell killing mediated by DNA double strand breaks and clustered fragmentation, that is largely independent of oxygenation status, cell cycle stage, and dose rate, due to the high linear energy transfer (LET; ˜100 keV/μm) of alpha particles. Currently, aside from ²²³Ra, the most widely investigated radionuclide for clinical TAT is ²²⁵Ac.

Current methods of imaging the biodistribution of AER are sub-optimal, primarily due to limitations on administered activity. This has several related knock-on effects: (i) AER biodistributions are unknown; (ii) absorbed dose distributions for AER are unknown; (iii) the correspondence between AER biodistributions and those of potential imaging surrogates is unknown. Thus, our ability to anticipate normal tissue toxicity and prescribe personalized treatment is compromised. Accurate quantitative imaging of the AER biodistribution can help remedy this situation and advance the field.

There is urgent need to be able to convert the very noisy, low-count scintigraphic images produced from low activities of AER into quantitative images of activity distribution that are adequate for reliable dosimetry estimation. The systems and methods discussed herein include a novel approach to improve quantitative TAT imaging. Current TAT images are of insufficient quality to implement the type of personalized, dosimetry-based therapy that is anticipated to improve patient outcomes. Potential surrogate biomarkers can be valuable if they can be shown to accurately represent the AER biodistribution. However, the degree of correspondence cannot be assessed without improved TAT imaging. The lack of such methods is a barrier to progress; preventing the optimization of personalized TAT and impeding the development of new AER.

The systems and methods described herein include a novel approach for the generation of accurate, corrected planar scintigraphic images of AER (CPSIAER) using low-count AER planar data and co-registered low-dose CT images acquired on SPECT/CT cameras. The obtained CPSIAER combined with CT can permit accurate AER activity estimation that may be used as input data for a dosimetry model, which can provide information on the radiation dose to organs/tumor. The systems and methods described herein can include a novel sparsity promoting reconstruction approach based on a physical model for planar AER imaging. Low-count ²²⁵Ac planar images and co-registered low-dose CT images acquired on SPECT/CT cameras, for example, as well as physical and digital phantoms, can be used.

Initial studies performed with simulated data, can show that the sparsity promoting methods can substantially improve quantitative and detective performance of scintigraphic imaging of AER. The novel approach may rely on a physical imaging model containing the system kernels and regularization. To control noise, a sparsity promoting regularization with envelope of the 10-norm can be used. The fixed-point proximity-operator approach (or other approaches) can be used to solve the resulting nonconvex minimization problem. The effects of noise on tumor/organ activity estimates may be accounted for with an ensemble mean squared error performance metric.

As a result of said novel approach, the direct quantitative imaging of AER and optimized personalized design of TAT including improved activity/fractionation schedule may become available. It can lead to higher likelihood of response and cure. It can also enable rational assessment of the utility of imaging AER surrogates.

Alpha-emitting radiopharmaceuticals (AER), such as ²²⁵Ac-PSMA-617 in the treatment of metastatic castration-resistant prostate cancer (mCRPC), show the exciting clinical potential of Targeted Alpha Therapy (TAT). TAT can be applied to the treatment of many other advanced-stage cancers including leukemia, melanoma, bladder cancer, glioma, and neuroendocrine tumors. There are two main benefits provided by alpha emitters: (i) The short range of α-particles in the tissue comparable, to a few cell diameters (<100 μm), which allows selective targeted cancer cell killing while avoiding off-target cytotoxicity, in contrast to external-beam radiation therapy or beta emitter-based therapy (e.g. with ⁹⁰Y, ¹³¹I, or ¹⁷⁷Lu³³), and (ii) The high energy (typically in the 4-9 MeV range) of alpha particles combined with much higher linear energy transfer (LET; 25-230 keV/μm) vs. β-particle (0.1-1 keV/μm) promotes cancer cell killing via DNA double strand breaks and clustered fragmentation, reducing or eliminating the dependence of cytotoxicity on oxygenation status, cell cycle stage, dose rate, and largely overcoming cellular radio-resistance. Currently, aside from ²²³Ra the most clinically investigated radionuclide for clinical TAT is ²²⁵Ac (T_{1/2}=9.9 d, pure α emitter with energies in the 5.8-8.4 MeV, 47-85 μm range in tissue). In most foreseeable circumstances, ²²⁵Ac exists in close-to-secular equilibrium with its radioactive progeny including ²²¹Fr (T_{1/2}=4.8 min, pure a emitter with 218 keV γ emission at 11.4% yield) and ²¹³Bi (T_{1/2}=46 min, mixed α/β emitter with 440 keV γ emission at 26.1% yield). ²²⁵Ac itself has no significant γ emission and can only be non-invasively imaged using the γ emissions of ²²¹Fr and/or ²¹³Bi. Clinical trials with ²²⁵Ac-PSMA-617 or ²²⁵Ac-DOTATATE in patients resistant to conventional radionuclide therapies with beta emitters have demonstrated the clinical potential of TAT, with objective responses and mild side effects reported. Clinical studies with ²²⁵Ac-labeled SSRT- and PSMA-targeting small molecules are ongoing worldwide.

Because currently AER biodistributions are mostly unknown, the absorbed dose distributions for AER are unknown. Thus, safety (dose to normal tissues/organs) and efficacy (dose to tumor) of AER are difficult to access. Patient selection and some aspects of risk stratification and predictive biodistribution for AER can be accomplished via pretreatment quantitative imaging with single photon or positron-emitting surrogates of AER. For example, ⁶⁸Ga-PSMA quantitative PET/CT imaging may be performed to select patients for ²²⁵Ac-PSMA and for baseline, interval and restaging quantification of the extent of disease. However, the correspondence between AER biodistributions and those of potential imaging surrogates remains unknown and limits the ability to determine optimal AER dose for a specific patient. Further, surrogate imaging is not “real-time” and has to be repeated each time information is needed. It cannot be used to assess off target anomalous distribution (e.g., extravasation, contamination, labeling issues). Direct imaging of AER is desirable but at present provides poor quality images. Thus, the ability to anticipate normal tissue toxicity and prescribe personalized treatment is compromised.

AER are difficult to image, especially those that decay via alpha-emitting progeny (e.g. ²²⁵Ac), because only very low activities can be administered (˜100 kBq/kg). This is a consequence of the high radiobiological potency of AER and consequently only low-count gamma camera-based imaging is possible. Current SOC methods produce images that are very noisy and difficult to quantify. Recently, hybrid protocols combining whole-body planar imaging with quantitative abdominal SPECT/CT for ²²⁵Ac-PSMA has been reported, but the utility of SPECT for ²²⁵Ac remains anecdotal. To ensure that TAT is both effective and safe, in vivo molecular imaging that enables the efficacious verification of AER biodistribution and therapeutic index is necessary. Consequently, there is an urgent unmet need to develop appropriate methods for low-count gamma camera image processing. The absence of such methods represents a barrier to progress in the field of TAT; hindering the personalization of treatment to provide maximum benefit and minimize toxicity and impeding the development of new AERs.

The systems and methods discussed herein can address this unmet need by implementation of a novel regularized model-based approach to reconstruct an accurate corrected planar scintigraphic AER biodistribution image (CPSIAER) and verify its superiority using a task-based image quality (IQ) assessment approach. The proposed model can be based on an anterior-posterior conjugate image pair acquired by a single photon emission computed tomography (SPECT/CT) physical system. It may comprise modeling of the collimator (including septal penetration), spatial and energy resolution, and attenuation and scatter (from the accompanying CT). Noise may be controlled by means of sparsity promoting regularization, which utilizes the envelope of the 10-norm (env-10). A preconditioned fixed-point algorithm (PFA) can be used to solve the resulting nonconvex minimization problem. A task-based methodology is applied for assess IQ assessment.

Because of the extremely low activity of administered AER, too few counts are acquired to generate clinically acceptable whole-body tomographic images. As a result, planar whole-body imaging is performed instead of SPECT. Unfortunately, the planar images are very difficult to quantify and are very noisy. To address these weaknesses, the systems and methods described herein can use accurate image formation models and/or advanced sparsity promoting regularization methods to obtain a CPSIAER from an anterior-posterior conjugate whole-body planar image pair acquired by a SPECT/CT camera, and use co-registered CT data to correct for collimator distance-dependent spatial resolution, scatter, attenuation, and/or collimator-originated artifacts. The resultant higher-quality lower-noise quantitative images of the AER biodistributions combined with CT images of tumors/organs may be suitable for 3D dosimetry studies.

The systems and methods described herein can include a new approach for scintigraphic whole-body planar image processing in TAT. It may allow optimized personalized design of TAT including activity/fractionation schedule leading to higher likelihood of response and cure. It may also enable rational assessment of the utility of imaging surrogates, and is hypothesized to improve the therapeutic ratio of TAT. External co-registered CT can be used with older gamma cameras, not equipped with CT. The proposed systems/methods may not require vendor's support for implementation. Hence, they can be applied to any planar scintigraphic imaging studies.

Various embodiments of the systems and methods discussed here improve the computational properties of the AER imaging model. To control noise, approximate sparsity regularization via the env-10 can be used. In the past, such models were intractable due to 10-norm's discontinuous nature. However, the novel approach discussed herein (env-10) makes the use of 10-norm manageable via an efficient preconditioned fixed-point algorithm. Task-based IQ assessment may be used to evaluate improvement. The systems and methods may use a regularized image reconstruction approach for generating corrected planar images. Said systems and methods may include an implementation of a hybrid planar/CT data acquisition by means of SPECT/CT camera and exploitation of CT data for estimation of 3D dose distribution in tumors and organs from 2D planar scintigraphic data. The systems and methods may introduce an envelope of the 10-norm approximate sparsity metric for planar image reconstruction. The systems and methods may apply an innovative, efficient preconditioned fixed-point algorithm for solving the env-10 model.

The recent remarkable success of AER in the treatment of metastatic castration-resistant prostate cancer with ²²⁵Ac-PSMA-617 and other cancers has revealed the striking clinical potential of TAT. However, current methods of imaging the biodistribution of AER are sub-optimal due to very low administered activity (˜100 kBq/kg) resulting in low-count (very noisy) data necessitating planar scintigraphic imaging rather than SPECT. The SOC methods for processing the low-count planar imaging data cannot provide accurate information on AER and hence, on absorbed dose to organs/tumor. Also, the correspondence between AER biodistributions and those of potential imaging surrogates is unknown, compromising the ability to anticipate normal tissue toxicity and prescribe personalized treatment. To remedy this problem, a method for accurate quantitative imaging of the AER biodistribution is needed.

In various embodiments, sparsity promoting regularization can be used to control noise via the novel env-10 norm sparsity metric. In a low-count/high-noise AER, imaging regularization plays two roles: it reduces the ill-posedness of the problem and denoises the images. Ill-posedness is reduced by constraining the solution space. Noise reduction is achieved by using a transform to concentrate the image's meaningful information (energy) on only a few of its components. Noise contamination remains evenly spread out with small magnitude values that can be removed by a threshold, resulting in high accuracy with much less noise. To amplify the denoising performance of the systems/methods, a specially designed TF can be applied, based on a redundant multiscale discrete cosine transform and the env-10 norm, which behaves like the 11-norm near the origin and like the 10-norm elsewhere, as a tractable approximation of the 10-norm due to its continuity despite its nonconvexity. The env-10 norm has the added advantage that it can be unbiased, unlike the 11-norm that removes energy from the signal. The resulting non-smooth, non-convex minimization problem can be formulated as a fixed-point equation and iteratively solved.

The systems and methods discussed herein can include an energy dependent physical model of a gamma imaging SPECT/CT system with high-energy collimators. Photon transport through the collimator can also model septal penetration. Scatter may be estimated via a Monte Carlo kernel and/or scatter window fitting. Scintillation light and/or spatial resolution loss in the NaI (TI) crystal may be modeled. In certain embodiments, a fixed-point formulation of the TF-L0 model can be applied for fast convergence. The validated power-law model can be used to automate regularization weight selection specific to each patient and the administered activity. The regularization optimization procedure may be iterative.

It is noted that, in various embodiments, the functions performed by the systems, devices, and components depicted in, for example, FIGS. 1A-1D and 2 may be performed by a greater number of components or fewer components, and may be performed by other combinations of devices and systems. For example, the functions performed by one component as depicted may instead be performed by two or more components, and/or the functions performed by two or more components as depicted may instead be performed by one component. Similarly, functions may be redistributed among components, devices, and systems. For example, the functions performed by one combination of components, devices, and/or systems as depicted may instead be performed by another combination of components, devices, and/or systems.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It is noted that terms such as “approximately,” “substantially,” “about,” or the like may be construed, in various embodiments, to allow for insubstantial or otherwise acceptable deviations from specific values. In various embodiments, deviations of 20 percent may be considered insubstantial deviations, while in certain embodiments, deviations of 15 percent may be considered insubstantial deviations, and in other embodiments, deviations of 10 percent may be considered insubstantial deviations, and in some embodiments, deviations of 5 percent may be considered insubstantial deviations. In various embodiments, deviations may be acceptable when they achieve the intended results or advantages, or are otherwise consistent with the spirit or nature of the embodiments.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

1. A computing system comprising:
one or more processors; and
a non-transitory computer-readable medium having instructions stored thereon that when executed by the one or more processors cause the computing system to:
obtain, by the one or more processors, a plurality of planar scintigraphy images of a subject, wherein the plurality of planar scintigraphy images contain image artifacts caused by one or more physical processes;
generate, by the one or more processors, a corrected planar scintigraphy image (CPSI) corrected for the image artifacts by applying a planar scintigraphy image reconstruction model to the plurality of planar scintigraphy images, the planar scintigraphy image reconstruction model comprising a non-negativity constraint and being based on a regularization term and a fidelity term; and
present, by the one or more processors, the CPSI for evaluation of a condition of the subject, wherein presenting the CPSI comprises at least one of transmitting the CPSI to a computing device or displaying the CPSI on a display screen.

2. The computing system of claim 1, wherein the plurality of planar scintigraphy images comprise an anterior planar scintigraphy image or a posterior planar scintigraphy image.

3. The computing system of claim 1, wherein the one or more physical processes comprise gamma ray attenuation, gamma ray collimator penetration, or gamma ray scatter.

4. The computing system of claim 1, wherein obtaining the plurality of planar scintigraphy images comprises using a plurality of gamma ray detectors to generate the plurality of planar scintigraphy images.

5. The computing system of claim 1, wherein the regularization term corresponds to a total variation regularization for controlling noise.

6. The computing system of claim 1, wherein the regularization term corresponds to tight framelets based on a transform.

7. The computing system of claim 6, wherein the transform is a redundant multiscale discrete cosine transform.

8. The computing system of claim 1, wherein the planar scintigraphy image reconstruction model comprises a minimization operation based on the regularization term, the fidelity term, and the non-negativity constraint.

9. The computing system of claim 8, wherein the minimization operation is based on a divergence norm and a regularization parameter (λ).

10. The computing system of claim 1, wherein the planar scintigraphy image reconstruction model is based on a two-view single photon emission computed tomography (SPECT) physical model, optionally wherein the two-view SPECT physical model comprises an anterior view and a posterior view or optionally wherein generating the CPSI comprises: estimating an anterior/posterior (A/P) projection of activity bio-distribution using the two-view SPECT physical model as a constraint.

11. (canceled)

12. (canceled)

13. The computing system of claim 10, wherein the two-view SPECT physical model is determined according to:
wherein
$${\int }_{{J}^{3}} K ( x ; y ) f ( y ) dy = g ( x ) , x ∈ {J}^{3} , \\ x := ( {x}_{1} , {x}_{2} , {x}_{3} ) ,$$
y:=(y₁, y₂, y₃),
the g(x) describes the plurality of planar scintigraphy images,
the f(y) describes a 3D biodistribution, and
the K(x; y) describes a kernel of a region J:=[α, b].

14. The computing system of claim 13, wherein the K(x; y) is related to an attenuation map derived from computed tomography (CT).

15. The computing system of claim 13, wherein applying the planar scintigraphy image reconstruction model comprises determining:
$$\overline{f} ( {y}_{1} , {y}_{2} ) = {\int }_{J} f ( {y}_{1} , {y}_{2} , {y}_{3} ) {dy}_{3} subject to : \\ {min}_{f} \{ {‖ {\int }_{{J}^{3}} K ( \cdot ; y ) f ( y ) dy - g ( \cdot ) ‖}_{KL} + λ {‖ f ‖}_{TV ( {J}^{3} )} + {ι}_{+} ( f ) \} ,$$
wherein the ƒ̅(y₁, y₂) describes the CPSI,
the ∥∫_{J}_{³}K(·;y)f(y)dy−g(·)∥_{KL} corresponds to a divergence norm corresponding to the fidelity term,
the λ∥ƒ∥_{TV(J}_{³}₎ corresponds to a total variation regularization for controlling noise corresponding to the regularization term, and
the l₊(ƒ) corresponds to an indicator function imposing the non-negativity constraint.

16. The computing system of claim 15, wherein the planar scintigraphy image reconstruction model is discretized according to:
ƒ̅=Σ_{y}_{₃}ƒ(y), subject to:
$${min}_{f} \{ {‖ Kf - g ‖}_{KL} + λ {‖ {B}_{3} f ‖}_{1} + {ι}_{+} ( f ) \} , {‖ Kf - g ‖}_{KL} = Kf - g log ( Kf ) ,$$
wherein the B₃ corresponds to a three dimensional first order gradient block matrix.

17. The computing system of claim 16, wherein the instructions further cause the computing system to apply the discretized planar scintigraphy image reconstruction model using a fixed point algorithm with higher order total variation regularization (HOTV) according to:
$$\{ \begin{matrix}{f}^{k + 1} = {prox}_{{ι}_{+}} \{ {f}^{k} - S [ {K}^{T} ( 1 - \frac{g}{{Kf}^{k}} ) + {B}_{3}^{T} {b}^{k} + {C}_{3}^{T} {c}^{k} ] \} \\ {b}^{k + 1} = {ρ}_{1} ( I - {prox}_{\frac{{λ}_{1}}{{ρ}_{1}} {φ}_{1}} ) ( \frac{{b}^{k}}{ρ} + {B}_{3} ( 2 {f}^{k + 1} - {f}^{k} ) ) \\ {c}^{k + 1} = {ρ}_{2} ( I - {prox}_{\frac{{λ}_{2}}{{ρ}_{2}} {φ}_{2}} ) ( \frac{{c}^{k}}{ρ} + {C}_{3} ( 2 {f}^{k + 1} - {f}^{k} ) )\end{matrix} ,$$
wherein the b corresponds to a first subgradient term,
the c corresponds to a second subgradient term,
the C₃ corresponds to a three dimensional second order gradient block matrix,
the φ₁ corresponds to a first order isotropic total variation norm,
the φ₂ corresponds to a second order isotropic total variation norm,
the λ₁ corresponds to a first regularization parameter,
the λ₂ corresponds to a second regularization parameter,
the S corresponds to a preconditioner,
the ρ₁ corresponds to a first algorithmic parameter, and
the ρ₂ corresponds to a second algorithmic parameter.

18. A method comprising:
obtaining, by a computing system, a plurality of planar scintigraphy images of a subject, wherein the plurality of planar scintigraphy images contain image artifacts caused by one or more physical processes;
generating, by the computing system, a corrected planar scintigraphy image (CPSI) corrected for the image artifacts by applying a planar scintigraphy image reconstruction model to the plurality of planar scintigraphy images, the planar scintigraphy image reconstruction model comprising a non-negativity constraint and being based on a regularization term and a fidelity term; and
presenting, by the computing system, the CPSI for evaluation of a condition of the subject, wherein presenting the CPSI comprises at least one of transmitting the CPSI to a computing device or displaying the CPSI on a display screen.

19. The method of claim 18, wherein at least one of:
the plurality of planar scintigraphy images comprise an anterior planar scintigraphy image or a posterior planar scintigraphy image; or
the one or more physical processes comprise gamma ray attenuation, gamma ray collimator penetration, or gamma ray scatter; or
obtaining the plurality of planar scintigraphy images comprises using a plurality of gamma ray detectors to generate the plurality of planar scintigraphy images; or
wherein the regularization term corresponds to a total variation regularization for controlling noise; or
wherein the regularization term corresponds to tight framelets based on a transform, wherein the transform is a redundant multiscale discrete cosine transform; or
wherein the planar scintigraphy image reconstruction model comprises a minimization operation based on the regularization term, the fidelity term, and the non-negativity constraint, wherein the minimization operation is based on a divergence norm and a regularization parameter (λ).

20-26. (canceled)

27. The method of claim 18, comprising:
determining, according to the generated CPSI, a dosage of radiation administered to the subject that minimizes a risk of toxicity to non-cancerous tissue, while optimizing treatment for cancerous tissue.

28. The method of claim 18, wherein the planar scintigraphy image reconstruction model is based on a two-view single photon emission computed tomography (SPECT) physical model, and at least one of:
(A) wherein the two-view SPECT physical model comprises an anterior view and a posterior view; or
(B) wherein generating the CPSI comprises estimating an anterior/posterior (A/P) projection of activity bio-distribution using the two-view SPECT physical model as a constraint; or
(C) wherein the method comprises:
(1) determining the two-view SPECT physical model according to:
$${\int }_{{J}^{3}} K ( x ; y ) f ( y ) dy = g ( x ) , x ∈ {J}^{3} ,$$
wherein x:=(x₁, x₂, x₃),
y:=(y₁, y₂, y₂),
the g(x) describes the plurality of planar scintigraphy images,
the ƒ(y) describes a 3D biodistribution,
the K(x; y) describes a kernel of a region J:=[α, b];
(2) determining the K(x; y) according to an attenuation map derived from computed tomography (CT), wherein applying the planar scintigraphy image reconstruction model comprises determining:
7̅1̅ (y₁, y₂)=∫_{J} ƒ(y₁, y₂, y₃)dy₃ subject to;
$${min}_{f} \{ {‖ {\int }_{{J}^{3}} K ( \cdot ; y ) f ( y ) dy - g ( \cdot ) ‖}_{KL} + λ {‖ f ‖}_{TV ( {J}^{3} )} + {ι}_{+} ( f ) \} ,$$
wherein:
the ƒ̅(y₁, y₂) describes the CPSI,
the ∥∫_{J}_{³}K(·;y)ƒ(y)dy−g(·)∥_{KL} corresponds to a divergence norm corresponding to the fidelity term,
the λ∥ƒ∥_{TV(J}_{³}₎ corresponds to a total variation regularization for controlling noise corresponding to the regularization term, and
the l₊(ƒ) corresponds to an indicator function imposing the non-negativity constraint;
(3) discretizing the planar scintigraphy image reconstruction model according to:
ƒ̅=Σ_{y}_{^{s}}ƒ(y), subject to:
$${min}_{f} \{ {‖ Kf - g ‖}_{KL} + λ {‖ {B}_{3} f ‖}_{1} + {ι}_{+} ( f ) \} , {‖ Kf - g ‖}_{KL} = Kf - g log ( Kf ) ,$$
wherein the B₃ corresponds to a three dimensional first order gradient block matrix; and
(4) applying the discretized planar scintigraphy image reconstruction model using a fixed point algorithm with higher order total variation regularization (HOTV) according to:
$$\{ \begin{matrix}{f}^{k + 1} = {prox}_{{ι}_{+}} \{ {f}^{k} - S [ {K}^{T} ( 1 - \frac{g}{{Kf}^{k}} ) + {B}_{3}^{T} {b}^{k} + {C}_{3}^{T} {c}^{k} ] \} \\ {b}^{k + 1} = {ρ}_{1} ( I - {prox}_{\frac{{λ}_{1}}{{ρ}_{1}} {φ}_{1}} ) ( \frac{{b}^{k}}{ρ} + {B}_{3} ( 2 {f}^{k + 1} - {f}^{k} ) ) \\ {c}^{k + 1} = {ρ}_{2} ( I - {prox}_{\frac{{λ}_{2}}{{ρ}_{2}} {φ}_{2}} ) ( \frac{{c}^{k}}{ρ} + {C}_{3} ( 2 {f}^{k + 1} - {f}^{k} ) )\end{matrix} ,$$
wherein the b corresponds to a first subgradient term,
the c corresponds to a second subgradient term,
the C₃ corresponds to a three dimensional second order gradient block matrix,
the φ₁ corresponds to a first order isotropic total variation norm,
the φ₂ corresponds to a second order isotropic total variation norm,
the λ₁ corresponds to a first regularization parameter,
the λ₂ corresponds to a second regularization parameter,
the S corresponds to a preconditioner,
the ρ₁ corresponds to a first algorithmic parameter, and
the ρ₂ corresponds to a second algorithmic parameter.

29-35. (canceled)

36. The method of claim 18, further comprising using the CPSI to evaluate the condition of the subject.
